(19) United States
(12) Patent Application Publication
(10) Pub. No.: US 2003/0138429 A1
(43) Pub. Date: Jul. 24, 2003

(54) Compositions and methods for inhibiting metastasis

(76) Inventors: Salvatore V. Pizzo, Bahama, NC (US); Mario Gonzalez-Gronow, Chapel Hill, NC (US)

Correspondence Address: WOMBLE CARLYLE SANDRIDGE & RICE, PLLC, Post Office Box 7037, Atlanta, GA, 30357-0037, US

(21) Appl. No.: 10/153,428

(22) Filed: May 22, 2002

Related U.S. Application Data
(60) Provisional application No. 60/292,621, filed on May 22, 2001.

(51) Int. Cl. A61K 48/00; A61K 38/43; A61K 39/395; A61K 9/127

(52) U.S. Cl. 424/155.1; 424/450; 424/94.1; 514/44

(57) ABSTRACT

Compounds, compositions and methods for inhibiting metastasis, and screening methods for identifying compounds are disclosed. The compounds bind to CD26 and/or plasminogen, and when so bound, inhibit the Ca⁺² signaling cascade that results in the formation of MMP-9. When the compounds directly bind to CD26 in a manner that inhibits the signaling cascade, they inhibit metastasis. When the compounds enhance the ability of angiostatin to bind to CD26 and inhibit the signaling cascade, they are angiostatin allosteric promoters. The compounds can also bind to CD26 in a manner which inhibits the binding of ADA to CD26/DPP IV, and such compounds used in methods for inhibiting deamination of adenosine. The compounds can be, for example, antibodies, antibody fragments, enzymes, peptides, nucleic acids such as oligonucleotides, or small molecules. The antibodies can be monoclonal, humanized, or polyclonal antibodies. The compounds can be conjugated to or combined with various cytotoxic agents and/or labeled compounds. Methods for inhibiting tumor metastasis can be used to treat patients suffering from such tumors.

### RELATED APPLICATION

This application is a non-provisional application claiming the benefit of Provisional Application Serial No. 60/292,621, filed May 22, 2001, the content of which is hereby incorporated in its entirety.

### FIELD OF THE INVENTION

This application is generally in the area of compositions and methods for inhibiting metastasis.

### BACKGROUND OF THE INVENTION

The ability of tumor cells to display invasive behavior involves the activation of mechanisms that provide for focal degradation of the basement membrane in which the cells reside. These mechanisms involve the expression of new receptors, which in addition to enabling the tumor cell to escape from the strict regulation that components of the basement membrane exert in the physiology of the normal cell, they provide protection from attack by immunocompetent cells, thereby assuring their viability in the circulation. One such receptor is dipeptidyl peptidase IV (CD26/DPP IV).

Most of the functional properties of CD26/DPP IV have been elucidated in T lymphocytes, where the molecule is physically associated in its extracellular domain with CD45 and may serve as a receptor for adenosine deaminase (ADA), both of which may be of importance during T cell activation and signal transduction [1-3]. The association of CD26/DPP IV with ADA not only permits a rapid metabolization of adenosine, which in excess is toxic to lymphocytes, but may also serve as a docking protein for the attachment of T cells to tissues or cells also expressing CD26/DPP IV on their surface [4].

Apart from lymphoid tissues, CD26/DPP IV is found lining the blood vessels of most human tissues [6] where it has been hypothesized to play a critical role in downregulating blood coagulation by preventing the attachment of fibrin clots to the capillary walls [6]. In the liver, the molecule participates in tissue destruction and regeneration processes [7]. In the kidney, the molecule is found preferentially in glomeruli [7]. In lung endothelium, CD26/DPP IV is an adhesion molecule for lung-metastatic rat breast and prostate carcinoma cells [8].

The physiological role of CD26/DPP IV in tissues lacking the proteins to which it normally associates in T lymphocytes has been extensively studied in hepatocarcinoma cell lines [9]. Stimulation of these cells with anti-CD26 mAbs induces apoptosis [9]. By contrast, a similar stimulation of CD26-Jurkat T cells with the same mAbs protects these cells from apoptosis after human immunodeficiency virus infection [10], suggesting that CD26/DPP IV contribution to cell physiology depends on the complex receptor context and exerts different functions in different cell types.

In human rheumatoid synovial fibroblasts [11] and prostate cancer cell lines 1-LN, PC-3 and DU-145 [12], CD26/DPP IV is a receptor for plasminogen (Pg) and is colocalized with the urinary-type plasminogen activator receptor (uPAR) [11,12]. Pg binds to this receptor via its oligosaccharide chains to a peptide comprising the DPP IV primary sequence L₃₁₃QWLRRI [13]. CD26/DPP IV is also a receptor for fibronectin (FN) [14]. Binding of FN is mediated by a polypeptide comprising the FN primary sequence L₁₇₆₈ TSRPA [15].

It would be advantageous to have new compositions and methods to add to the arsenal of therapies available for inhibiting tumor metastasis. It would also be advantageous to have new methods for identifying such compositions and methods. The present invention provides such compositions and methods.

### SUMMARY OF THE INVENTION

The present invention is directed to compounds, compositions and methods for inhibiting tumor metastasis, and results from the discovery that angiostatin binds to CD26 in a manner that inhibits plasminogen from binding to CD26, and when so bound, inhibits the Ca⁺² signaling cascade which leads to the expression of MMP-9. The invention is also directed to compositions and methods for inhibiting adenosine deamination by ADA.

In one embodiment, the compounds bind to CD26 in a manner which inhibits the ability of plasminogen to bind to CD26 (CD26 antagonists). When so bound, they also inhibit the Ca⁺² signaling cascade which leads to the expression of MMP-9.

In another embodiment, the compounds bind to the oligosaccharide chains on plasminogen that would otherwise bind to CD26 (plasminogen antagonists). The bound oligosaccharide chains then are inhibited from binding to CD26, which also inhibits the Ca⁺² signaling cascade which leads to the expression of MMP-9, which in turn inhibits tumor metastasis.

In a third embodiment, the compounds (ADA antagonists) bind to the CD26/DPP IV primary region that includes the polypeptide L₃₄₀ VAR, or to a position that sterically interferes with this region. The polypeptide L₃₄₀ VAR is responsible for binding to adenosine deaminase (ADA). When the polypeptide is bound by the ADA antagonists, the cells are exposed to the cytotoxic effects of adenosine and ADA is prevented from serving as a possible anchor between circulating tumor cells and CD26/DPP IV lining the blood vessels.

The compounds can be, for example, antibodies, antibody fragments, enzymes, proteins, peptides, nucleic acids such as oligonucleotides, or small molecules. The antibodies can be, for example, monoclonal, humanized (chimeric) or polyclonal antibodies, and can be prepared, for example, using conventional techniques. The compounds can be conjugated to various cytotoxic agents and/or labeled compounds.

The compounds can be included in various compositions, for example, compositions suitable for intravenous, intramuscular, topical, local, intraperitoneal, or other forms of administration. They can be targeted to capillary beds by incorporating them into appropriately sized microparticles or liposomes that remain lodged in capillary beds and release the compounds at a desired location.

The methods can be used to treat metastatic tumors. The methods involve administering effective amounts of suitable anti-metastasis compounds (i.e., CD26 antagonists, angiostatin allosteric promoters, plasminogen antagonists and/or ADA antagonists) and/or compositions including the compounds to patients in need of treatment. Effective anti-metastasis amounts are amounts effective to inhibit at least a significant amount of the metastasis that would otherwise occur in the absence of treatment.

Screening methods can be used to identify compounds useful in these methods. The screening methods can identify compounds that bind to CD26 and/or plasminogen, in particular, compounds that bind to the plasminogen binding site (L₃₁₃ QWLRRI) and/or the ADA binding site (L₃₄₀ VAR), or to positions that sterically interfere with these sites, as well as determining the activity of the compounds once bound.

Combinatorial libraries of compounds, for example, phage display peptide libraries, small molecule libraries and oligonucleotide libraries can be screened. Compounds that bind to CD26 or plasminogen, can be identified, for example, using affinity binding studies, or using other screening techniques known to those of skill in the art. The effect of the compounds once bound to CD26 or plasminogen can be determined, for example, by evaluating the level of plasminogen binding to CD26, MMP-9 synthesis, adenosine deaminase function, inhibition of Matrigel invasion by 1-LN cells, and the degree of tumor metastasis.

### BRIEF DESCRIPTION OF THE DRAWINGS

FIG. 1. Binding of individual Pg 2 glycoforms to 1-LN human prostate tumor cells. Increasing concentrations of ¹²⁵I-labeled Pg 2α (◯), Pg 2β (●), Pg 2γ (Δ), Pg 2δ (▴), Pg 2ε (□), or Pg 2φ (▪) were added to 1-LN cells. Molecules of ligand bound were calculated after subtraction of non-specific binding measured in the presence of 50-fold excess of nonlabeled ligands as described under Experimental Procedures. Data represent the mean±SD from experiments performed in triplicate.

FIG. 2. Inhibition of binding of individual Pg 2 glycoforms to 1-LN cells. (A) Cells were incubated in serum-free RPMI 1640 with a single concentration (0.1 μM) of ¹²⁵I-labeled Pg 2α (◯), Pg 2β (●), Pg 2γ (Δ), Pg 2δ (▴), or Pg 2ε (□) in the presence of increasing concentrations of 6-AHA, (B) cells were incubated in serum-free RPMI 1640 with a single concentration (0.1 μM) or ¹²⁵I-labeled Pg 2α (◯), Pg 2β (●), Pg 2γ (Δ), Pg 2δ (▴), or Pg 2ε (□) in the presence of increasing concentrations of L-lactose. Data represent the mean±SD from experiments performed in triplicate.

FIG. 3. Fluorescence-activated cell-sorter analyses of 1-LN cells. (A) Cells were incubated with a FITC-conjugated anti-human DPP IV murine mAb (solid line) or a FTIC-conjugated isotype control murine Mab (stippled line). (B) Cells were incubated with a FTIC-conjugated anti-human GPIIIa (β₃) murine Mab (solid line) or a FTIC-conjugated isotype control murine Mab (stippled line). (C) Cells were incubated with an anti-human FAP α, Mab F19, followed by a FTIC-conjugated anti-mouse IgG (solid line) or a FTIC-conjugated isotype control murine Mab (stippled line).

FIG. 4. Binding of individual Pg 2 glycoforms to immobilized DPP IV isolated from 1-LN cell membranes. (A) 96-well plates were coated with DPP IV (1 μg/ml) from 1-LN cell membranes. Increasing concentrations of ¹²⁵I-labeled Pg 2α (◯), Pg 2β (●), Pg 2γ (Δ), Pg 2δ (▴), Pg 2ε (□), or Pg 2φ (▪) were added to triplicate wells and incubated at 22° C. for 1 h. Bound Pg was quantified as described under Experimental Procedures. Data represent the means±SD of experiments performed in triplicate. Inset, 10% SDS-PAGE of purified DPP IV (5 μg) under reducing conditions. Lane 1, Coomassie Brilliant Blue R-250 stained gel; lane 2, blot incubated with anti-DPP IV IgG (mAb 236.3) followed by reaction with an alkaline phosphatase-conjugated secondary IgG. (B) Binding inhibition of ¹²⁵I-labeled Pg 2γ (Δ), Pg 2δ (▴), or Pg 2ε (□) (0.1 μM) to immobilized DPP IV by increasing concentrations of L-lactose. Bound Pg was quantified as described under Experimental Procedures.

FIG. 5. [Ca²⁺]ᵢ response of 1-LN cells to the binding of individual Pg 2 glycoforms. Cells were preloaded with 4 μM of Fura-2/AM for 20 min at 37° C. and changes in [Ca²⁺]ᵢ were measured as described under Experimental Procedures. Arrows indicate the times of addition of each individual Pg 2 glycoform (0.1 μM). (A) Stimulation by Pg 2α. (B) Stimulation by Pg 2β. (C) Stimulation by Pg 2γ. (D) Stimulation by Pg 2δ. (E) Stimulation by Pg 2ε. (F) Stimulation by Pg 2φ. (G) Stimulation by Pg 2γ in the presence of L-lactose (100 mM). (H) Stimulation by Pg 2δ in the presence of L-lactose (100 mM). (I) Stimulation by Pg 2ε in the presence of L-lactose (100 mM).

FIG. 6. Analysis of MMP-9 purified from 1-LN cell conditioned medium. Protein samples (5 μg) were resolved in a continuous 10% SDS-polyacrylamide gel and electroblotted to a nitrocellulose membrane as described under Experimental Procedures. Lane 1, Coomassie Brilliant blue R-250 blue stained gel. Lane 2, electroblot incubated with an anti-MMP-9 mAb. Lane 3, gelatinolytic activity of the proteins. The amino-terminal sequence of the major protein bands is shown at the left side of lane 1.

FIG. 7. Effect of Pg 2 glycoforms on the expression of MMP-9 by 1-LN cells. Cell monolayers in 48 well culture plates (1×10⁶ cells/well) were incubated with serum-free RPMI 1640 in the absence or presence of purified Pg 2 glycoforms (0.1 μM) in a volume of 0.3 ml at 37° C. for 24 h. Both zymographic and identification of MMP-9 by Western-blot analyses in conditioned medium were performed as described under Experimental Procedures. (A) Zymographic analysis of conditioned medium (50 μl) of cells incubated with each individual Pg 2 glycoform. (B) Western blot analysis of conditioned media (50 μl) of cells incubated with each individual Pg 2 glycoform. (C) Zymographic analysis of conditioned medium (50 μl) of cells incubated with each individual Pg 2 glycoform in the presence of L-lactose (100 mM). (D) Western Blot analysis of conditioned medium (50 μl) of cells incubated with each individual Pg 2 glycoform in the presence of L-lactose (100 mM). Each individual Pg 2 glycoform is identified at the base of each lane.

FIG. 8. Effect of anti-DPP IV IgG on the expression of MMP-9 induced by highly sialylated Pg 2 glycoforms. Cell monolayers in 48 well culture plates (1.7×10⁶ cells/well) were incubated in serum-free RPMI 1640 with each individual Pg 2 glycoform (0.1 μM) in the absence or presence of anti-DPP IV IgG (50 μg/ml) in a volume of 0.3 ml at 37° C. for 24 h. (A) Zymographic analysis of conditioned medium (50 μl) from cells incubated with Pg 2γ, Pg 2δ or Pg 2ε in the absence (lanes 1, 2, and 3, respectively) or presence of anti-DPP IV IgG (lanes 4, 5 and 6, respectively). (B) Western blot analysis of conditioned medium from cells incubated with anti-DPP IV IgG and highly sialylated Pgs 2γ, Pg 2δ, or Pg 2ε. The blots were reacted with anti-MMP-9 IgG. Each individual Pg 2 glycoform is identified at the base of each lane.

FIG. 9. Pg induced MMP-9 mRNA expression in cultured 1-LN cells. 1-LN cell monolayers in 48 well culture plates (1.7×10⁶ cells/well) were incubated in serum-free RPMI 1640 with each individual Pg 2 glycoform (0.1 μM) in a volume of 0.3 ml at 37° C. for 24 h. Isolation of total cytoplasmic RNA and measurements of MMP-9 mRNA by RT-PCR was performed as described under Experimental Procedures. Ethidium bromide-stained gels were photographed and analyzed by laser densitometric scanning. MMP-9 mRNA levels were expressed as relative MMP-9 mRNA/GAPDH mRNA ratios. Values represent the mean±SD of three separate experiments, each carried out in duplicate.

### DETAILED DESCRIPTION OF THE INVENTION

The following description includes the best presently contemplated mode of carrying out the invention. This description is made for the purpose of illustrating the general principles of the inventions and should not be taken in a limiting sense.

Compounds, compositions and methods for promoting or inhibiting tumor metastasis and/or inhibiting adenosine deamination by ADA are disclosed. In one embodiment, the compounds bind to CD26 in a manner which inhibits the ability of plasminogen to bind to CD26 (CD26 antagonists). When so bound, they also inhibit the Ca⁺² signaling cascade which leads to the expression of MMP-9, which in turn inhibits tumor metastasis.

In another embodiment, the compounds bind to the oligosaccharide chains on plasminogen that would otherwise bind to CD26 or to other positions on plasminogen that sterically interfere with the binding of CD26 to plasminogen (plasminogen antagonists). By inhibiting the binding of plasminogen to CD26, the Ca⁺² signaling cascade which leads to the expression of MMP-9 is also inhibited.

In a third embodiment, the compounds bind to CD26/DPP IV primary region that includes the polypeptide L₃₄₀ VAR, which is responsible for binding to adenosine deaminase (ADA), and when so bound, thus exposing the cell to the cytotoxic effects of adenosine and preventing ADA from serving as a possible anchor between circulating tumor cells and CD26/DPP IV lining the blood vessels (ADA antagonists).

Also disclosed are screening methods for identifying compounds that bind to CD26 in a manner that inhibits the Ca⁺² signaling cascade that results in the formation of MMP-9, as well as compounds that enhance the ability of angiostatin to bind to CD26 (angiostatin allosteric promoters). Methods for determining whether such compounds bind to CD26, in particular, to the plasminogen and/or ADA binding sites, are also disclosed. Screening methods for identifying compounds that bind to plasminogen in a manner that inhibits CD26 binding, as well as identifying compounds that bind to the polypeptide L₃₄₀ VAR which is responsible for binding to ADA are also disclosed.

The present invention is based on the discovery that angiostatin binds to CD26 and, through this binding, inhibits the Ca⁺² signaling cascade the results in the formation of MMP-9, which in turn inhibits tumor metastasis, and that the CD26/DPP IV primary region, including the polypeptide L₃₄₀ VAR is responsible for binding to ADA. Compounds that bind to CD26 and/or plasminogen and that also inhibit the Ca⁺² signaling cascade can also inhibit tumor metastasis. Compounds that bind to the polypeptide L₃₄₀ VAR or that bind to a site such that the interaction of the polypeptide L₃₄₀ VAR with ADA is sterically hindered expose the tumor cells to the cytotoxic effects of adenosine and also prevent ADA from serving as an anchor between circulating tumor cells and CD26/DPP IV.

The binding of plasminogen (Pg) to CD26/DPP IV on the surface of human prostate cancer 1-LN cells initiates a Ca⁺² signaling cascade that mediates synthesis and secretion of gelatinase B (MMP-9) [12]. This process facilitates the invasive capacity of 1-LN cells of membranes coated with Matrigel. However, as discussed in more detail in the Example, when the cells are incubated with Pg in the presence of anti-DPP IV monoclonal antibodies (mAbs), which prevent the interaction of Pg with DPP IV, the invasion of Matrigel by 1-LN cells is completely abolished. A similar inhibitory effect is observed when cells are incubated with Pg in the presence of L-lactose, a sugar which prevents binding of Pg oligosaccharide chains to CD26/DPP IV. In both of these experiments, the lack of invasive activity was correlated with a decrease in the expression of MMP-9 by the cells.

Experiments performed with angiostatin, a kringle containing polypeptide fragment of Pg, which is a potent inhibitor of angiogenesis, tumor growth and metastasis [18-19], also produced total inhibition of Matrigel invasion by 1-LN cells. Similarly, the FN peptide L₁₇₆₈ TSRPA inhibited Pg-induced Matrigel invasion by 1-LN cells in a dose-dependent manner. Taken together, these experiments suggest a central role of CD26/DPP IV in the invasive capacity of 1-LN prostate cancer cells.

These findings are not only useful as a diagnostic tool, but also in deciding effective therapeutic strategies. These strategies include the following criteria:

1. Development of agents to prevent Pg binding to CD26/DPP IV, in particular, compounds that bind to the primary sequence L₃₁₃QWLRRI, which is the site of attachment of Pg oligosaccharide chains. The compounds can be either mAbs or other compounds that are capable of binding this polypeptide, for example oligosaccharides analogous to the ones found in Pg. In both cases, the interaction is inhibited, thus preventing the Ca⁺² signaling cascade which leads to the expression of MMP-9.

2. The use of angiostatin or the FN polypeptide L₁₇₆₈ TSRPA, both of which inhibit Pg binding to CD26/DPP IV, thereby preventing activation of Pg on the cell surface. Both these agents will not only prevent the tumor from growing, they will also inhibit colonization of distant normal tissues.

3. The development of mAbs or other compounds that bind the CD26/DPP IV primary region comprising the polypeptide L₃₄₀ VAR, which is responsible for binding to ADA. This would not only expose the tumor cell to the cytotoxic effects of adenosine, but will prevent ADA from serving as a possible anchor between circulating tumor cells and CD26/DPP IV lining the blood vessels.

Definitions

The following definitions will be helpful in understanding the compositions and methods described herein.

As used herein, the term “tumor metastasis” is defined as the spreading of a tumor by escaping from the basement membrane in which the tumor cells reside.

The term “angiostatin” refers to a proteolytic fragment of plasminogen, and includes at least one kringle, and preferably, at least three kringles, from plasminogen. Angiostatin is a potent inhibitor of angiogenesis and the growth of tumor cell metastases (O'Reilly et al., Cell 79:315328 (1994)). All anti-metastatic forms of angiostatin are intended to be included within the definition of angiostatin as used herein.

Angiostatin has a specific three dimensional conformation that is defined by the kringle region of the plasminogen molecule. (Robbins, K. C., “The plasminogen/plasmin enzyme system” Hemostasis and Thrombosis, Basic Principles and Practice, 2nd Edition, ed. by Colman, R. W. et al. J. B. Lippincott Company, pp. 340357, 1987). There are five such kringle regions, which are conformationally related motifs and have substantial sequence homology in the amino terminal portion of the plasminogen molecule.

A variety of silent amino acid substitutions, additions, or deletions can be made in the above identified kringle fragments, which do not significantly alter the fragments' endothelial cell inhibiting activity. Each kringle region of the angiostatin molecule contains approximately 80 amino acids and contains 3 disulfide bonds. Antiangiogenic angiostatin can include a varying amount of amino or carboxy-terminal amino acids from the inter-kringle regions and may have some or all of the naturally occurring disulfide bonds reduced. Angiostatin may also be provided in an aggregate, non-refolded, recombinant form.

Angiostatin can be generated in vitro by limited proteolysis of plasminogen, as taught by Sottrup Jensen et al., Progress in Chemical Fibrinolysis and Thrombolysis 3:191209 (1978), the contents of which are hereby incorporated by reference for all purposes. This results in a 38 kDa plasminogen fragment (Va179Pro353). Angiostatin can also be generated in vitro by reducing plasmin (Gately et al., PNAS 94:1086810872 (1997)) and in Chinese hamster ovary and human fibrosarcoma cells (Stathakis et al., JBC 272(33):20641.20645 (1997)).

Angiostatin may also be produced from recombinant sources, from genetically altered cells implanted into animals, from tumors, and from cell cultures as well as other sources. Angiostatin can be isolated from body fluids including, but not limited to, serum and urine. Recombinant techniques include gene amplification from DNA sources using the polymerase chain reaction (PCR), and gene amplification from RNA sources using reverse transcriptase/PCR.

The term “CD26 antagonist” as used herein refers to a compound that binds to CD26, and when so bound, inhibits the binding of plasminogen to CD26, which in turn inhibits the Ca⁺² signaling cascade that results in the formation of MMP-9, which in turn inhibits tumor metastasis. While angiostatin is an example of a suitable CD26 antagonist, angiostatin has a relatively short half life in vivo, and other compounds with similar binding affinity for CD26 but with longer half lives may be preferred.

The term “plasminogen antagonist” as used herein refers to a compound that binds to plasminogen, in one embodiment, to the oligosaccharide chains that would otherwise bind CD26, and when so bound, inhibits the binding of plasminogen to CD26, which in turn inhibits the Ca⁺² signaling cascade that results in the formation of MMP-9, which in turn inhibits tumor metastasis.

The term “ADA antagonist” as used herein refers to a compound that binds to the polypeptide L340 VAR on CD26/DPP IV in a manner that inhibits the binding of CD26 to ADA, or that binds in a position that sterically hinders this binding, which in turn inhibits the ability of ADA to destroy adenosine and also which inhibits the ability of ADA to serve as an anchor between circulating tumor cells and the CD26/DPP IV lining the blood vessels.

The term “angiostatin allosteric promoter” as used herein refers to a compound that does directly bind to CD26, but enhances the ability of angiostatin to bind to CD26.

The terms “a”, “an” and “the” as used herein are defined to mean “one or more” and include the plural unless the context is inappropriate.

As employed herein, the phrase “active agent” or “active compound” refers to CD26 antagonists, plasminogen antagonists, ADA antagonists and angiostatin allosteric promoters. Examples of suitable biologically active compounds/agents include antibodies, antibody fragments, enzymes, peptides, nucleic acids, and small molecules.

As used herein, peptide is defined as including less than or equal to 100 amino acids and protein is defined as including 100 or more amino acids.

Unless defined otherwise, all technical and scientific terms used herein have the same meaning as commonly understood by one having ordinary skill in the art. Although other materials and methods similar or equivalent to those described herein can be used in the practice or testing of the present invention, as would be apparent to practitioners in the art, the preferred methods and materials are now described.

I. Methods of Inhibiting Tumor Metastasis

Tumor metastasis can be inhibited by administering an effective amount of a suitable CD26 and/or plasminogen antagonist (for example, antibodies, antibody fragments, and/or small molecules) to a patient in need of such treatment. Angiostatin allosteric promoters can also be administered, alone or in combination with the CD26 antagonists. The compounds can either inhibit tumor metastasis on their own, or allosterically enhance the ability of angiostatin (or CD26 or plasminogen antagonists) to inhibit metastasis. The methods can be used to treat patients suffering from metastatic tumors. ADA antagonists can also be administered to prevent the deamination of adenosine.

The therapeutic and diagnostic methods described herein typically involve administering an effective amount of the compositions described herein to a patient. The exact dose to be administered will vary according to the use of the compositions and on the age, sex and condition of the patient, and can readily be determined by the treating physician. The compositions may be administered as a single dose or in a continuous manner over a period of time. Doses may be repeated as appropriate.

The compositions and methods can be used to treat metastasis of a variety of solid tumors, including colorectal carcinoma, gastric carcinoma, signet ring type, esophageal carcinoma, intestinal type, mucinous type, pancreatic carcinoma, lung carcinoma, breast carcinoma, renal carcinoma, bladder carcinoma, prostate carcinoma, testicular carcinoma, ovarian carcinoma, endometrial carcinoma, thyroid carcinoma, liver carcinoma, larynx carcinoma, mesothelioma, neuroendocrine carcinomas, neuroectodermal tumors, melanoma, gliomas, neuroblastomas, sarcomas, leiomyosarcoma, MFII, fibrosarcoma, liposarcoma, MPNT, chondrosarcoma, and lymphomas.

II. Compounds for Inhibiting Tumor Metastasis and/or Adenosine Deamination

Various compounds, including various antibodies, can bind to CD26 and inhibit plasminogen binding (CD26 antagonists). Various other compounds, including various antibodies, do not bind to CD26 but enhance the ability of CD26 antagonists to inhibit plasminogen binding angiostatin allosteric promoters). Still other compounds bind to plasminogen and interfere with the binding of plasminogen to CD26. Yet other compounds bind to CD26 in a manner that interferes with the binding of CD26/DPP IV to ADA.

The mere fact that the compounds bind to CD26 or plasminogen does not determine their ultimate effect on tumor metastasis. The compounds, when so bound, also must inhibit the binding of plasminogen to CD26, which in turn inhibits the Ca+2 signaling cascade that results in the formation of MMP-9, which in turn inhibits tumor metastasis.

The activity of the compounds once bound can be readily determined using the assays described herein. The compounds described herein are not limited to a particular molecular weight. The compounds can be large molecules (i.e., those with a molecular weight above about 1000) or small molecules (i.e., those with a molecular weight below about 1000). Examples of suitable types of compounds include antibodies, antibody fragments, enzymes, peptides and oligonucleotides.

A. Antibodies

Antibodies can be generated that:

a) bind to CD26, and, in particular, to the plasminogen binding portion of CD26, which portion has been identified as the primary sequence L₃₁₃ QWLRRI, the site of attachment of plasminogen oligosaccharide chains,

b) bind to plasminogen in such a manner that the binding of plasminogen to CD26 is inhibited, for example, antibodies that bind to the plasminogen oligosaccharide chains involved in such binding, and by blocking the ability of the polysaccharide chains to bind CD26, inhibit the ability of plasminogen to bind to CD26.

c) bind to CD26 in a manner that inhibits the binding of CD26/DPP IV to ADA.

Polyclonal antibodies can be used, provided their overall effect is decreased tumor metastasis. However, monoclonal antibodies are preferred. Humanized (chimeric) antibodies can be even more preferred.

The antibodies may not and need not bind in exactly the same way as angiostatin or the FN polypeptide L₁₇₆₈ TSRPA. Angiostatin has several potential binding portions (possibly involving the various kringles), and the antibodies likely do not include portions that mimic each of these binding portions. However, the antibodies may inhibit CD26, plasminogen or ADA binding by sterically interfering with and/or binding to all or part of the actual binding site(s).

Antibodies, in particular, monoclonal antibodies (mAbs) have been developed against CD26 and plasminogen that can be used either to directly inhibit metastasis or to target cytotoxic drugs or radioisotopic or other labels to sites of metastasis. The antibodies can be extremely specific. Furthermore, unlike other lines of research which have produced cancer cell specific mAbs to target cytotoxic drugs to tumors, these mAbs are prepared against host antigens (i.e., CD26 which is not found in normal cells). This approach has the major advantage that generation of “resistant” variants of the tumor cannot occur and, in theory, one mAb can be used to treat all solid tumors.

Antibody Preparation

The term “antibody” refers to a polypeptide substantially encoded by an immunoglobulin gene or immunoglobulin genes, or fragments thereof, that specifically binds and recognizes an analyte (antigen, in this case CD26, plasminogen and/or various binding domains thereof, preferably human CD26 and/or plasminogen). Immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit includes a tetramer. Each tetramer is composed of two identical pairs of polypeptide chains, each pair having one “light” (about 25 kD) and one “heavy” chain (about 50-70 kD). The N-terminus of each chain has a variable region of about 100 to 110 or more amino acids primarily responsible for antigen recognition. The terms “variable light chain” (or “VL”) and “variable heavy chain” (or “VH”) refer to these light and heavy chains, respectively.

Antibodies exist, for example, as intact immunoglobulins or as a number of well characterized antigen-binding fragments produced by digestion with various peptidases. For example, pepsin digests an antibody below the disulfide linkages in the hinge region to produce an F(ab′)₂ fragment, a dimer of Fab which itself is a light chain joined to VH-CH1 by a disulfide bond. The F(ab′)₂ fragment can be reduced under mild conditions to break the disulfide linkage in the hinge region, thereby converting the F(ab′)₂ dimer into an Fab′ monomer. The Fab′ monomer is essentially an Fab with part of the hinge region (see Fundamental Immunology, Third Edition, W. E. Paul (ed.), Raven Press, N.Y. (1993), the contents of which are hereby incorporated by reference). While various antibody fragments are defined in terms of the digestion of an intact antibody, one of ordinary skill in the art will appreciate that such fragments can be synthesized de novo either chemically or by using recombinant DNA methodology. Thus, the term antibody, as used herein, also includes antibody fragments, such as a single chain antibody, an antigen binding F(ab′)₂ fragment, an antigen binding Fab′ fragment, an antigen binding Fab fragment, an antigen binding Fv fragment, a single heavy chain or a chimeric (humanized) antibody. Such antibodies can be produced by modifying whole antibodies or synthesized de novo using recombinant DNA methodologies.

The CD26 and/or plasminogen (including fragments, derivatives, and analogs thereof) can be used as an immunogen to generate antibodies which immunospecifically bind such immunogens. Such antibodies include but are not limited to polyclonal antibodies, monoclonal antibodies, chimeric antibodies, single chain antibodies, antigen binding antibody fragments (e.g., Fab, Fab′, F(ab′)₂, Fv, or hypervariable regions), and mAb or Fab expression libraries. In some embodiments, polyclonal and/or monoclonal antibodies to CD26, plasminogen or the fragments, derivatives and/or analogs thereof are produced. In yet other embodiments, fragments of the CD26 and/or plasminogen that are identified as immunogenic are used as immunogens for antibody production.

Various procedures known in the art can be used to produce polyclonal antibodies. Various host animals (including, but not limited to, rabbits, mice, rats, sheep, goats, camels, and the like) can be immunized by injection with the antigen, fragment, derivative or analog. Various adjuvants can be used to increase the immunological response, depending on the host species. Such adjuvants include, for example, Freund's adjuvant (complete and incomplete), mineral gels such as aluminum hydroxide, surface active substances such as lysolecithin, pluronic polyols, polyanions, peptides, oil emulsions, keyhole limpet hemocyanins, dinitrophenol, and other adjuvants, such as BCG (bacille Calmette-Guerin) and Corynebacterium parvum.

Any technique that provides for the production of antibody molecules by continuous cell lines in culture can be used to prepare monoclonal antibodies directed toward the CD26, plasminogen, fragments thereof or binding portions thereof. Such techniques include, for example, the hybridoma technique originally developed by Kohler and Milstein (see, e.g., Nature 256:495-97 (1975)), the trioma technique (see, e.g., Hagiwara and Yuasa, Hum. Antibodies Hybridomas 4:15-19 (1993); Hering et al., Biomed. Biochim. Acta 47:211-16 (1988)), the human B-cell hybridoma technique (see, e.g., Kozbor et al., Immunology Today 4:72 (1983)), and the EBV-hybridoma technique to produce human monoclonal antibodies (see, e.g., Cole et al., In: Monoclonal Antibodies and Cancer Therapy, Alan R. Liss, Inc., pp. 77-96 (1985)). Human antibodies can be used and can be obtained by using human hybridomas (see, e.g., Cote et al., Proc. Natl. Acad. Sci. USA 80:2026-30 (1983)) or by transforming human B cells with EBV virus in vitro (see, e.g., Cole et al., supra).

“Chimeric” or “humanized” antibodies (see, e.g., Morrison et al., Proc. Natl. Acad. Sci. USA 81:6851-55 (1984); Neuberger et al., Nature 312:604-08 (1984); Takeda et al., Nature 314:452-54 (1985)) can also be prepared. Such chimeric antibodies are typically prepared by splicing the non-human genes for an antibody molecule specific for antigen together with genes from a human antibody molecule of appropriate biological activity. It can be desirable to transfer the antigen binding regions (e.g., Fab′, F(ab′)₂, Fab, Fv, or hypervariable regions) of non-human antibodies into the framework of a human antibody by recombinant DNA techniques to produce a substantially human molecule. Methods for producing such “chimeric” molecules are generally well known and described in, for example, U.S. Pat. Nos. 4,816,567; 4,816,397; 5,693,762; and 5,712,120; PCT Patent Publications WO 87/02671 and WO 90/00616; and European Patent Publication EP 239 400 (the disclosures of which are incorporated by reference herein). Alternatively, a human monoclonal antibody or portions thereof can be identified by first screening a cDNA library for nucleic acid molecules that encode antibodies that specifically bind to the CD26 and/or plasminogen or fragments or binding domains thereof according to the method generally set forth by Huse et al. (Science 246:1275-81 (1989)), the contents of which are hereby incorporated by reference. The nucleic acid molecule can then be cloned and amplified to obtain sequences that encode the antibody (or antigen-binding domain) of the desired specificity. Phage display technology offers another technique for selecting antibodies that bind to the CD26, plasminogen, fragments, derivatives or analogs thereof and binding domains thereof. (See, e.g., International Patent Publications WO 91/17271 and WO 92/01047; Huse et al., supra.) Techniques for producing single chain antibodies (see, e.g., U.S. Pat. Nos. 4,946,778 and 5,969,108) can also be used. An additional aspect of the invention utilizes the techniques described for the construction of a Fab expression library (see, e.g., Huse et al., supra) to allow rapid and easy identification of monoclonal Fab fragments with the desired specificity for antigens, fragments, derivatives, or analogs thereof.

Antibodies that contain the idiotype of the molecule can be generated by known techniques. For example, such fragments include but are not limited to, the F(ab′)₂ fragment which can be produced by pepsin digestion of the antibody molecule, the Fab′ fragments which can be generated by reducing the disulfide bridges of the F(ab′)₂ fragment, the Fab fragments which can be generated by treating the antibody molecule with papain and a reducing agent, and Fv fragments. Recombinant Fv fragments can also be produced in eukaryotic cells using, for example, the methods described in U.S. Pat. No. 5,965,405 (the disclosure of which is incorporated by reference herein).

Antibody screening can be accomplished by techniques known in the art (e.g., ELISA (enzyme-linked immunosorbent assay)). In one example, antibodies that recognize a specific domain of an antigen can be used to assay generated hybridomas for a product which binds to polypeptides containing that domain. Antibodies specific to a domain of an antigen are also provided.

Antibodies against the CD26 and/or plasminogen (including fragments, derivatives and analogs and binding domains thereof) can be used for passive antibody treatment, according to methods known in the art. The antibodies can be produced as described above and can be polyclonal or monoclonal antibodies and administered intravenously, enterally (e.g., as an enteric coated tablet form), by aerosol, orally, transdermally, transmucosally, intrapleurally, intrathecally, or by other suitable routes.

Small amounts of humanized antibody can be produced in a transient expression system in CHO cells to establish that they bind to cells expressing CD26. Stable cell lines can then be isolated to produce larger quantities of purified material.

The binding affinity of murine and humanized antibodies can be determined using the procedure described by Krause et al., Behring Inst. Mitt., 87:5667 (1990). Briefly, antibodies can be labeled with fluorescein using fluorescein isothiocyanate (FITC), and then incubated with HUVEC cells for two hours on ice in PBS containing fetal calf serum (FCS) and sodium azide. The amount of fluorescence bound per cell can be determined in a FACScan and calibrated using standard beads. The number of molecules of antibody that had bound per cell at each antibody concentration can be established and used to generate Scatchard plots. Competition assays can be performed by FACScan quantitation of bound antibody after incubating the cells with a standard quantity of the murine antibody together with a dilution series of the humanized variants.

B. Multivalent Compounds

Multivalent compounds are defined herein as compounds that include more than one moiety capable of being attached to the CD26 and/or plasminogen or binding domains thereof or fragments, analogs and derivatives thereof.

In one embodiment, the multifunctional compound includes at least one protein and/or peptide chain. Alternatively, the compound can include small molecules with a plurality of moieties with bind properties as described above.

C. High Throughput Screening Methods for mAb Libraries

High throughput monoclonal antibody assays can be used to determine the binding affinities of the antibodies to the targets, and also identify which antibodies act as antagonists of the targets. The assays can evaluate, for example, increased or decreased MPP-9 expression, the binding of CD26 to plasminogen, Matrigel invasion, and/or the levels or the degree of tumor metastasis. Suitable assays are described, for example, in the Examples. Similar high throughput assays can be used to evaluate the properties of small molecule libraries.

Similar screening methods can be used to identify other classes of compounds useful in the methods described herein. Combinatorial libraries of compounds, for example, phage display peptide libraries, small molecule libraries and oligonucleotide libraries can be screened. Compounds that bind to the targets can be identified, for example, using competitive binding studies.

D. Antibody/Drug Conjugates

Antibodies raised against the targets, and, in particular, monoclonal antibodies, can be conjugated to a drug. The drug/antibody complex can then be administered to a patient, and the antibody will bind to the targets in a manner that delivers a relatively high concentration of the drug to the desired tissue or organ. In some embodiments, the binding of the drug to the antibody is in a biodegradable linkage, so that the drug is released over time. In other embodiments, the drug remains attached to the antibody.

Anti-cancer drugs are an example of drugs that can be conjugated to the antibodies. For example, the antibodies can be conjugated with QFA, which is an antifolate, or with calicheamycin, adriamycin, bleomycin or vincamycin, which are anti-tumor antibiotics that cleave the double stranded DNA of tumor cells. Additional tumor treating compounds that can be coupled to the antibodies include BCNU, streptozoicin, vincristine, ricin, radioisotopes, and 5-fluorouracil and other anti-cancer nucleosides.

In vivo xenograft studies can be used to show that inhibition of tumor metastasis as well as direct tumor inhibition with limited normal tissue damage can be obtained with antibodies conjugated to these anti-cancer drugs. The antibody/drug conjugates can be used to target compounds directly to tumors that might otherwise be too toxic when administered systemically.

The conjugates are most advantageously used in combination with targeted drug delivery methods, for example, by placing the compounds in liposomes or other microparticles of an appropriate size such that they lodge in capillary beds around tumors and release the compounds at the tumor site. Alternatively the compounds can be injected directly into or around the site of a tumor, for example, via injection or catheter delivery. Such methods minimize any undesirable systemic effects.

Oligonucleotides with free, reactive hydroxy, amine, carboxy or thiol groups at either the 3′ or 5′ end can be conjugated to free reactive groups on antibodies using conventional coupling chemistry, for example, using heterobifunctional reagents such as SPDP. The 3′ or 5′ end of the oligonucleotide can be enzymatically labeled, for example, with ³²P as tracer for DNA. The final product can be tested for cell binding activity and protein and bound oligonucleotide concentrations. Depending on the activity of the oligonucleotides, the conjugates can be used for therapeutic or diagnostic purposes.

The antibodies (or other compounds that bind to the targets) can be conjugated with photosensitizers such as porphyrins and used in targeted photodynamic therapy. After the compositions are administered and allowed to bind to the targets, the photodynamic therapy can be conducted by irradiation with light at a suitable wavelength for a suitable amount of time.

Antibodies that bind to the targets can also be covalently or ionically coupled to various markers, and used to detect the presence of tumors. This generally involves administering a suitable amount of the antibody to the patient, waiting for the antibody to bind to the targets at or around a tumor site, and detecting the marker. Suitable markers are well known to those of skill in the art, and include for example, radioisotopic labels, fluorescent labels and the like, and detection methods for these markers are also well known to those of skill in the art. Examples of suitable detection techniques include positron emission tomography, autoradiography, flow cytometry, radioreceptor binding assays, and immunohistochemistry.

Generally, a background concentration of the compounds will be observed in locations throughout the body. However, a higher, detectable concentration will be observed in locations where a tumor is present. The label can be detected, and, accordingly, the tumors can be detected.

E. Small Molecules

As used herein, small molecules are defined as molecules with molecular weights below about 2000, except in the case of oligonucleotides that can be considered small molecules if their molecular weight is less than about 10,000 (about 30mer or less). Many companies currently generate libraries of small molecules, and high throughput screening methods for evaluating small molecule libraries to identify compounds that bind particular receptors are well known to those of skill in the art. Combinatorial libraries of small molecules can be screened and suitable compounds for use in the methods described herein can be identified using routine experimentation. One example of a suitable small molecule library is a phage display library. Another such library is a library including random oligonucleotides, typically with sizes less than about 100mers. The SELEX process can be used to screen such oligonucleotide libraries (including DNA, RNA and other types of genetic material, and also including natural and non-natural base pairs) for compounds that have suitable binding properties, and other assays can be used to determine the effect of the compounds on tumor metastasis.

The SELEX method is described in U.S. Pat. No. 5,270,163 to Gold et al. Briefly, a candidate mixture of single stranded nucleic acids with regions of randomized sequence can be contacted with the targets and those nucleic acids having an increased affinity to the targets can be partitioned from the remainder of the candidate mixture. The partitioned nucleic acids can be amplified to yield a ligand enriched mixture.

F. Peptide Phage Display Libraries

One technique that is useful for identifying peptides that bind to targets is phage display technology, as described, for example, in Phage Display of Peptides and Proteins: A Laboratory Manual; Edited by Brian K. Kay et al. Academic Press San Diego, 1996, the contents of which are hereby incorporated by reference for all purposes.

Phage peptide libraries typically include numerous different phage clones, each expressing a different peptide, encoded in a single stranded DNA genome as an insert in one of the coat proteins. In an ideal phage library the number of individual clones would be 20ⁿ, where “n” equals the number of residues that make up the random peptides encoded by the phage. For example, if a phage library was screened for a seven residue peptide, the library in theory would contain 20⁷ (or 1.28×10⁹) possible 7 residue sequences. Therefore, a 7-mer peptide library should contain approximately 10⁹ individual phage.

Methods for preparing libraries containing diverse populations of various types of molecules such as antibodies, peptides, polypeptides, proteins, and fragments thereof are known in the art and are commercially available (see, for example, Ecker and Crooke, Biotechnology 13:351360 (1995), and the references cited therein, the contents of each of which is incorporated herein by reference for all purposes). One example of a suitable phage display library is the Ph.D.7 phage display library (New England BioLabs Cat #8100), a combinatorial library consisting of random peptide 7-mers. The Ph.D.7 phage display library consists of linear 7-mer peptides fused to the pIII coat protein of M13 via a GlyGlyGlySer flexible linker. The library contains 2.8×10⁹ independent clones and is useful for identifying targets requiring binding elements concentrated in a short stretch of amino acids.

Phage clones displaying peptides that are able to bind to the targets are selected from the library. The sequences of the inserted peptides are deduced from the DNA sequences of the phage clones. This approach is particularly desirable because no prior knowledge of the primary sequence of the target protein is necessary, epitopes represented within the target, either by a linear sequence of amino acids (linear epitope) or by the spatial juxtaposition of amino acids distant from each other within the primary sequence (conformational epitope) are both identifiable, and peptidic mimotopes of epitopes derived from non-proteinaceous molecules such as lipids and carbohydrate moieties can also be generated.

A library of phage displaying potential binding peptides can be incubated with immobilized targets to select clones encoding recombinant peptides that specifically bind the immobilized targets. The phages can be amplified after various rounds of biopanning (binding to the immobilized targets) and individual viral plaques, each expressing a different recombinant protein, or binding peptide, can then be expanded to produce sufficient amounts of peptides to perform a binding assay.

Phage selection can be conducted according to methods known in the art and according to manufacturers' recommendations. The “target” proteins, CD26 and/or plasminogen, and, in particular, the L₃₁₃ QWLRRI peptide and/or the L₃₄₀ VAR polypeptide, can be coated overnight onto high binding plastic plates or tubes in humidified containers. In a first round of panning, approximately 2×10¹¹ phage can be incubated on the protein-coated plate for 60 minutes at room temperature while rocking gently. The plates can then be washed using standard wash solutions. The binding phage can then be collected and amplified following elution using the target protein. Secondary and tertiary pannings can be performed as necessary.

Following the last screening, individual colonies of phage-infected bacteria can be picked at random, the phage DNA isolated and then subjected to dideoxy sequencing. The sequence of the displayed peptides can be deduced from the DNA sequence.

III. Compositions

Therapeutic, prophylactic and diagnostic compositions containing the compounds described herein typically include one or more active compounds together with a pharmaceutically acceptable excipient, diluent or carrier for in vivo use. Such compositions can be readily prepared by mixing the active compound(s) with the appropriate excipient, diluent or carrier.

Any suitable dosage may be administered. The type of metastatic tumor to be treated, the compound, the carrier and the amount will vary widely depending on body weight, the severity of the condition being treated and other factors that can be readily evaluated by those of skill in the art. Generally a dosage of between about 1 milligrams (mg) per kilogram (kg) of body weight and about 100 mg per kg of body weight is suitable.

A dosage unit may include a single compound or mixtures thereof with other compounds or other anti-cancer agents. The dosage unit can also include diluents, extenders, carriers and the like. The unit may be in solid or gel form such as pills, tablets, capsules and the like or in liquid form suitable for oral, rectal, topical, intravenous injection or parenteral administration or injection into or around the tumor.

The compounds are typically mixed with a pharmaceutically acceptable carrier. This carrier can be a solid or liquid and the type is generally chosen based on the type of administration being used.

The compounds can be administered via any suitable route of administration that is effective in the treatment of the particular metastatic tumor-mediated disorder that is being treated. Treatment may be oral, rectal, topical, parenteral or intravenous administration or by injection into the tumor and the like. It is believed that parenteral treatment by intravenous, subcutaneous, or intramuscular application of the compounds, formulated with an appropriate carrier, additional cancer inhibiting compound or compounds or diluents to facilitate administration, will be the preferred method of administering the compounds.

The compounds can be incorporated into a variety of formulations for therapeutic administration. More particularly, the compounds can be formulated into pharmaceutical compositions by combination with appropriate, pharmaceutically acceptable carriers or diluents, and may be formulated into preparations in solid, semi-solid, liquid or gaseous forms, such as tablets, capsules, pills, powders, granules, dragees, gels, slurries, ointments, solutions, suppositories, injections, inhalants and aerosols. As such, administration of the compounds can be achieved in various ways, including oral, buccal, rectal, parenteral, intraperitoneal, intradermal, transdermal, intratracheal, etc., administration. Moreover, the compounds can be administered in a local rather than systemic manner, for example via injection of the compound directly into a solid tumor, often in a depot or sustained release formulation. In addition, the compounds can be administered in a targeted drug delivery system, for example, in a liposome coated with the antibodies described herein. Such liposomes will be targeted to and taken up selectively by the tumor.

In addition, the compounds can be formulated with common excipients, diluents or carriers, and compressed into tablets, or formulated as elixirs or solutions for convenient oral administration, or administered by the intramuscular or intravenous routes. The compounds can be administered transdermally, and can be formulated as sustained release dosage forms and the like.

The compounds can be administered alone, in combination with each other, or they can be used in combination with other known compounds (e.g., other anti-cancer drugs). For instance, the compounds can be used in conjunctive therapy with known anti-angiogenic chemotherapeutic and/or antineoplastic agents (e.g., vinca alkaloids, antibiotics, antimetabolites, platinum coordination complexes, etc.). For instance, the compounds can be used in conjunctive therapy with a vinca alkaloid compound, such as vinblastine, vincristine, taxol, etc.; an antibiotic, such as adriamycin (doxorubicin), dactinomycin (actinomycin D), daunorubicin (daunomycin, rubidomycin), bleomycin, plicamycin (mithramycin) and mitomycin (mitomycin C), etc.; an antimetabolite, such as methotrexate, cytarabine (AraC), azauridine, azaribine, fluorodeoxyuridine, deoxycoformycin, mercaptopurine, etc.; or a platinum coordination complex, such as cisplatin (cis-DDP), carboplatin, etc. In pharmaceutical dosage forms, the compounds may be administered in the form of their pharmaceutically acceptable salts, or they may also be used alone or in appropriate association, as well as in combination with other pharmaceutically active compounds.

Suitable formulations for use in the present invention are found in Remington's Pharmaceutical Sciences (Mack Publishing Company, Philadelphia, Pa., 17th ed. (1985)), which is incorporated herein by reference. Moreover, for a brief review of methods for drug delivery, see, Langer, Science 249:1527-1533 (1990), which is incorporated herein by reference. The pharmaceutical compositions described herein can be manufactured in a manner that is known to those of skill in the art, i.e., by means of conventional mixing, dissolving, granulating, dragee-making, levigating, emulsifying, encapsulating, entrapping or lyophilizing processes. The following methods and excipients are merely exemplary and are in no way limiting.

For injection, the compounds can be formulated into preparations by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. Preferably, the compounds can be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining with pharmaceutically acceptable carriers that are well known in the art. Such carriers enable the compounds to be formulated as tablets, pills, dragees, capsules, emulsions, lipophilic and hydrophilic suspensions, liquids, gels, syrups, slurries, suspensions and the like, for oral ingestion by a patient to be treated. Pharmaceutical preparations for oral use can be obtained by mixing the compounds with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas, or from propellant-free, dry-powder inhalers. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds are preferably formulated for parenteral administration by injection, e.g., by bolus injection or continuous infusion. Formulations for injection may be presented in unit dosage form, e.g., in ampules or in multidose containers, with an added preservative. The compositions may take such forms as suspensions, solutions or emulsions in oily or aqueous vehicles, and may contain formulator agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter, carbowaxes, polyethylene glycols or other glycerides, all of which melt at body temperature, yet are solidified at room temperature.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Alternatively, other delivery systems for hydrophobic pharmaceutical compounds may be employed. Liposomes and emulsions are well known examples of delivery vehicles or carriers for hydrophobic drugs. In a presently preferred embodiment, long-circulating, i.e., stealth, liposomes are employed. Such liposomes are generally described in Woodle, et al., U.S. Pat. No. 5,013,556, the contents of which are hereby incorporated by reference.

The compounds can be encapsulated in a vehicle such as liposomes that facilitates transfer of the bioactive molecules into the targeted tissue, as described, for example, in U.S. Pat. No. 5,879,713 to Roth et al., the contents of which are hereby incorporated by reference. The compounds can be targeted by selecting an encapsulating medium of an appropriate size such that the medium delivers the molecules to a particular target. For example, encapsulating the compounds within microparticles, preferably biocompatible and/or biodegradable microparticles, which are appropriate sized to infiltrate, but remain trapped within, the capillary beds and alveoli of the lungs can be used for targeted delivery to these regions of the body following administration to a patient by infusion or injection.

In a preferred embodiment, the liposome or microparticle has a diameter which is selected to lodge in particular regions of the body. For example, a microparticle selected to lodge in a capillary will typically have a diameter of between 10 and 100, more preferably between 10 and 25, and most preferably, between 15 and 20 microns. Numerous methods are known for preparing liposomes and microparticles of any particular size range. Synthetic methods for forming gel microparticles, or for forming microparticles from molten materials, are known, and include polymerization in emulsion, in sprayed drops, and in separated phases. For solid materials or preformed gels, known methods include wet or dry milling or grinding, pulverization, classification by air jet or sieve, and the like.

Microparticles can be fabricated from different polymers using a variety of different methods known to those skilled in the art. The solvent evaporation technique is described, for example, in E. Mathiowitz, et al., J. Scanning Microscopy, 4, 329 (1990); L. R. Beck, et al., Fertil. Steril., 31, 545 (1979); and S. Benita, et al., J. Pharm. Sci., 73, 1721 (1984). The hot-melt microencapsulation technique is described by E. Mathiowitz, et al., Reactive Polymers, 6, 275 (1987). The spray drying technique is also well known to those of skill in the art. Spray drying involves dissolving a suitable polymer in an appropriate solvent. A known amount of the compound is suspended (insoluble drugs) or co-dissolved (soluble drugs) in the polymer solution. The solution or the dispersion is then spray-dried. Microparticles ranging between 1-10 microns are obtained with a morphology which depends on the type of polymer used. Microparticles made of gel-type polymers, such as alginate, can be produced through traditional ionic gelation techniques. The polymers are first dissolved in an aqueous solution, mixed with barium sulfate or some bioactive agent, and then extruded through a microdroplet forming device, which in some instances employs a flow of nitrogen gas to break off the droplet. A slowly stirred (approximately 100-170 RPM) ionic hardening bath is positioned below the extruding device to catch the forming microdroplets. The microparticles are left to incubate in the bath to allow sufficient time for gelation to occur. Microparticle particle size is controlled by using various size extruders or varying either the nitrogen gas or polymer solution flow rates. Particle size can be selected according to the method of delivery which is to be used, typically IV injection, and where appropriate, entrapment at the site where release is desired. Liposomes are available commercially from a variety of suppliers. Alternatively, liposomes can be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound or its monophosphate, diphosphate, and/or triphosphate derivatives are then introduced into the container. The container is then swirled by hand to free lipid material from the sides of the container and to disperse lipid aggregates, thereby forming the liposomal suspension.

The monoclonal antibodies specific for the targets as described herein can optionally be conjugated to liposomes and the delivery can be targeted in this manner. In addition, targeting of a marker on abnormal tumor vasculature can be employed. The targeting moiety when coupled to a toxic drug or radioisotope will act to concentrate the drug where it is needed. Ligands for tumor-associated vessel markers can also be used. For example, a cell adhesion molecule that binds to a tumor vascular element surface marker can be employed. Liposomes and other drug delivery systems can also be used, especially if their surface contains a ligand to direct the carrier preferentially to the tumor vasculature. Liposomes offer the added advantage of shielding the drug from most normal tissues. When coated with polyethylene glycol (PEG) (i.e., stealth liposomes) to minimize uptake by phagocytes and with a tumor vasculature-specific targeting moiety, liposomes offer longer plasma half-lives, lower non-target tissue toxicity, and increased efficacy over non-targeted drug. Using the foregoing methods, the compounds can be targeted to the tumor vasculature to effect control of tumor progression or to other sites of interest (e.g., endothelial cells).

Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various types of sustained-release materials have been established and are well known by those skilled in the art. Sustained-release capsules may, depending on their chemical nature, release the compounds for a few days up to over 100 days. Such sustained release capsules typically include biodegradable polymers, such as polylactides, polyglycolides, polycaprolactones and copolymers thereof.

Pharmaceutical compositions suitable for use in the methods described herein include compositions wherein the active ingredients are contained in a therapeutically effective amount. The amount of composition administered will, of course, be dependent on the subject being treated, on the subject's weight, the severity of the affliction, the manner of administration and the judgment of the prescribing physician. Determination of an effective amount is well within the capability of those skilled in the art, especially in light of the detailed disclosure provided herein.

Therapeutically effective dosages for the compounds described herein can be estimated initially from cell culture assays. For example, a dose can be formulated in animal models to achieve a circulating concentration range that includes the IC₅₀ as determined in cell culture (i.e., the concentration of test compound that is lethal to 50% of a cell culture), or the IC₁₀₀ as determined in cell culture (i.e., the concentration of compound that is lethal to 100% of a cell culture). Such information can be used to more accurately determine useful doses in humans. Initial dosages can also be estimated from in vivo data.

Moreover, toxicity and therapeutic efficacy of the compounds described herein can be determined by standard pharmaceutical procedures in cell cultures or experimental animals, e.g., by determining the LD₅₀, (the dose lethal to 50% of the population) and the ED₅₀ (the dose therapeutically effective in 50% of the population). The dose ratio between toxic and therapeutic effect is the therapeutic index and can be expressed as the ratio between LD₅₀ and ED₅₀. Compounds which exhibit high therapeutic indices are preferred. The data obtained from these cell culture assays and animal studies can be used in formulating a dosage range that is not toxic for use in human. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. (See, e.g., Fingl et al., 1975, In: The Pharmacological Basis of Therapeutics, Ch. 1, p. 1).

Dosage amount and interval may be adjusted individually to provide plasma levels of the active compound which are sufficient to maintain therapeutic effect. Preferably, therapeutically effective serum levels will be achieved by administering multiple doses each day. In cases of local administration or selective uptake, the effective local concentration of the drug may not be related to plasma concentration. One having skill in the art will be able to optimize therapeutically effective local dosages without undue experimentation.

While the composition may be administered by routes other than intravenously (i.v.), intraveneous administration is preferred. This is because the target of the therapy is primarily tumor cells, which are located adjacent to vasculature feeding the tumors; and thus, administering the composition intravenously saturates the targeted vasculature much quicker than if another route of administration is used. Additionally, the intravenous route allows for the possibility of further targeting to specific tissues.

In one embodiment, a catheter is used to direct the composition directly to the location of the target tumor. For example, if the tumor is located in the liver, then the immunoconjugate or the unconjugated antibody or a fragment thereof may be delivered into the hepatic portal vein using a catheter. In this embodiment, systemic distribution of composition is minimized, further minimizing any potential side effects from the therapy.

IV. Screening Methods

Various screening methods can be used to determine the ability of compounds to inhibit tumor metastasis and/or the binding of CD26/DPP IV to ADA. In the methods described herein, although many compounds can bind to CD26 and/or plasminogen, the mere fact that they bind CD26 or plasminogen does not determine their ultimate effect on tumor metastasis or ADA binding. The screening methods can be used to determine the ultimate effect of the compounds, once bound, on the binding of CD26 with plasminogen and/or the binding of CD26/DPP IV with ADA.

Various screening methods can also be used to determine the activity of compounds bound to the targets. Examples of suitable screening methods include measuring MPP-9 synthesis, measuring Matrigel invasion, and measuring tumor metastasis.

The compounds can be evaluated using in vitro assays to determine their biological activity. These assays are familiar to those skilled in the art and include Matrigel invasion assays. The ability of a compound to inhibit metastasis in these assays would indicate that the compound is either able to mimic the interaction of angiostatin with CD26.

The biological activity of the compounds may also be tested in vivo. Examples of suitable assays include the B16B16 metastasis assay or the Lewis Lung Carcinoma primary tumor or metastasis assays. In such experiments, the activity of the compounds can be compared to that of angiostatin if desired. Suitable binding assays are described in more detail below.

V. Binding Assays

CD26 and/or plasminogen, or the isolated polypeptide targets L₃₄₀ VAR and L₃₁₃ QWLRRI can be present in a suitable media, can be expressed on the surface of a tumor cell, or can be expressed in a cell that has been engineered to express these polypeptides.

The binding assays described herein can use any truncated forms of the targets. Binding assays include cell-free assays in which one or more of the targets (or fusion proteins containing same) are incubated with a test compound (proteinaceous or non-proteinaceous) which, advantageously, bears a detectable label (e.g., a radioactive or fluorescent label). Following incubation, the targets, free or bound to test compound, can be separated from unbound test compound using any of a variety of techniques. For example, the targets can be bound to a solid support (e.g., a plate or a column) and washed free of unbound test compound. The amount of test compound bound to targets is then determined, for example, using a technique appropriate for detecting the label used (e.g., liquid scintillation counting and gamma counting in the case of a radiolabeled test compound or by fluorometric analysis).

Binding assays can also take the form of cell-free competition binding assays. In such assays, one or more of the targets are incubated with a compound known to interact with the targets, which compound, advantageously, bears a detectable label (e.g., a radioactive or fluorescent label). A test compound (proteinaceous or non-proteinaceous) is added to the reaction and assayed for its ability to compete with the known (labeled) compound for binding to the targets.

Free known (labeled) compound can be separated from bound known compound, and the amount of bound known compound determined to assess the ability of the test compound to compete. This assay can be formatted so as to facilitate screening of large numbers of test compounds by linking the targets to a solid support so that it can be readily washed free of unbound reactants. A plastic support, for example, a plastic plate (e.g., a 96 well dish), is preferred. The targets described above can be isolated from natural sources (e.g., membrane preparations) or prepared recombinantly or chemically. The targets can be prepared as fusion proteins using, for example, known recombinant techniques. Preferred fusion proteins include a GST (glutathione-S-transferase) moiety, a GFP (green fluorescent protein) moiety (useful for cellular localization studies) or a His tag (useful for affinity purification). The non-target moiety can be present in the fusion protein N-terminal or C-terminal to the targets, subunits thereof or binding domains thereof.

As indicated above, the targets can be present linked to a solid support, including a plastic or glass plate or bead, a chromatographic resin (e.g., Sepharose), a filter or a membrane. Methods for attaching proteins to such supports are well known in the art and include direct chemical attachment and attachment via a binding pair (e.g., biotin and avidin or biotin and streptavidin). Whether free or bound to a solid support, the targets can be unlabeled or can bear a detectable label (e.g., a fluorescent or radioactive label).

The binding assays also include cell-based assays in which targets are presented on a cell surface. Cells suitable for use in such assays include cells that naturally express CD26 and/or plasminogen and cells that have been engineered to express CD26 and/or plasminogen (or subunits thereof, binding domains thereof and/or fusion proteins comprising same). The cells can be normal or tumorigenic. Advantageously, cells expressing human CD26 are used. Examples of suitable cells include procaryotic cells (e.g., bacterial cells (e.g., E. coli)), lower eucaryotic cells, yeast cells (e g., hybrid kits from Promega (CG 1945 and Y190), and the strains YPH500 and BJ5457)) and higher eucaryotic cells (e.g., insect cells and mammalian cells such as human lung carcinoma cells (e.g., A549 cells)).

Cells can be engineered to express the targets by introducing into a selected host an expression construct comprising a sequence encoding the targets, or subunit thereof or binding domains thereof or fusion protein, operably linked to a promoter. A variety of vectors and promoters can be used. For example, pET-24a(+) (Novagen) containing a T7 promoter is suitable for use in bacteria, likewise, pGEX-5X-1. Suitable yeast expression vectors include pYES2 (Invitron). Suitable baculovirus expression vectors include p2Bac (Invitron). Suitable mammalian expression vectors include pBK/CMV (Stratagene). Introduction of the construct into the host can be effected using any of a variety of standard transfection/transformation protocols (see Molecular Biology, A Laboratory Manual, second edition, J. Sambrook, E. F. Fritsch and T. Maniatis, Cold Spring Harbor Press, 1989). Cells thus produced can be cultured using established culture techniques suitable for the involved host. Culture conditions can be optimized to ensure expression of the targets (or subunits, binding domains or fusion proteins thereof) encoding sequence. While for the cell-based binding assays the targets (or subunit, binding domain or fusion protein) can be expressed on a host cell membrane (e.g., on the surface of the host cell), for other purposes the encoding sequence can be selected so as to ensure that the expression product is secreted into the culture medium. The cell-based binding assays described herein can be carried out by adding test compound (advantageously, bearing a detectable (e.g., radioactive or fluorescent) label), to medium in which the targets (or subunits thereof, binding domains thereof or fusion proteins containing same) expressing cells are cultured, incubating the test compound with the cells under conditions favorable to binding and then removing unbound test compound and determining the amount of test compound associated with the cells.

The presence of the targets on a cell membrane (e.g., on the cell surface) can be identified using techniques such as those in the Examples that follow (e.g., the cell surface can be biotin labeled and the protein followed by a fluorescent tag). Membrane associated proteins (e.g., cell surface proteins) can also be analyzed on a Western blot and the bands subjected to mass spectroscopy analysis. For example, a fluorescently tagged antibody can be used, and the cells can then be probed with another fluorescently tagged protein. Each tag can be monitored at a different wavelength, for example, using a confocal microscope to demonstrate co-localization.

As in the case of the cell-free assays, the cell-based assays can also take the form of competitive assays wherein a compound known to bind the targets (and preferably labeled with a detectable label) is incubated with the targets (or subunits thereof, binding domains thereof or fusion proteins comprising same) expressing cells in the presence and absence of test compound. The affinity of a test compound for the targets can be assessed by determining the amount of known compound associated with the cells incubated in the presence of the test compound, as compared to the amount associated with the cells in the absence of the test compound.

A test compound identified in one or more of the above-described assays as being capable of binding to the targets can, potentially, inhibit tumor metastasis, cellular migration, proliferation and pericellular proteolysis. To determine the specific effect of any particular test compound selected on the basis of its ability to bind the targets, assays can be conducted to determine, for example, the effect of various concentrations of the selected test compound on activity, for example, cell (e.g., endothelial cell) metastasis.

Examples of types of assays that can be carried out to determine the effect of a test compound on tumor metastasis include the Lewis Lung Carcinoma assay (O'Reilly et al., Cell 79:315 (1994)) and extracellular migration assays (Boyden Chamber assay: Kleinman et al., Biochemistry 25:312 (1986) and Albini et al., Can. Res. 47:3239 (1987)).

Accordingly, the methods permit the screening of compounds for their ability to inhibit the binding of plasminogen to CD26. In addition to the various approaches described above, assays can also be designed so as to be monitorable colorometrically or using time-resolved fluorescence.

In another embodiment, the invention relates to compounds identified using the above-described assays as being capable of binding to CD26 and/or inhibiting the Ca⁺² signaling cascade that results in MMP-9 formation. Such compounds can include novel small molecules (e.g., organic compounds (for example, organic compounds less than 500 Daltons), and novel polypeptides, oligonucleotides, as well as novel natural products (preferably in isolated form) (including alkyloids, tannins, glycosides, lipids, carbohydrates and the like). Compounds that bind to CD26 can be used to inhibit metastasis, for example, in tumor bearing patients.

The compounds identified in accordance with the above assays can be formulated as pharmaceutical compositions.

VI. Kits

Kits suitable for conducting the assays described herein can be prepared. Such kits can include CD26, or the plasminogen and/or ADA binding domains thereof, or fusion proteins comprising same, and/or plasminogen. These components can bear a detectable label. The kit can include a CD26-specific or plasminogen-specific antibody.

The kit can include any of the above components disposed within one or more container means. The kit can further include ancillary reagents (e.g., buffers) for use in the assays. Diagnostic methods based on the assays for binding CD26 to plasminogen can be used to identify patients suffering from tumor metastasis. The demonstration that CD26 binding to plasminogen initiates the Ca⁺² signaling cascade, and the resulting availability of methods of identifying agents that can be used to inhibit the binding of CD26 and plasminogen, make it possible to determine which individuals will likely be responsive to particular therapeutic strategies. Treatment strategies for individuals suffering from tumor metastasis can be designed more effectively and with greater predictability of a successful result.

The present invention will be better understood with reference to the following non-limiting examples.

### EXAMPLE 1

### Interaction of Plasminogen with Dipeptidyl Peptidase IV Initiates a Signal Transduction Mechanism which Regulates Expression of Matrix Metalloproteinase-9 by Prostate Cancer Cells

Both plasminogen (Pg) activation and matrix metalloproteinases (MMPs) are involved in proteolytic degradation of extracellular matrix components, a requisite event for malignant cell metastasis. The highly invasive 1-LN human prostate tumor cell line synthesizes and secretes large amounts of Pg activators and MMPs. We demonstrate here that the Pg type 2 (Pg 2) receptor in these cells is composed primarily of the membrane glycoprotein dipeptidyl peptidase IV (DPP IV). Pg 2 has six glycoforms that differ in their sialic acid content. Only the highly sialylated Pg 2γ, Pg 2δ, and Pg 2ε glycoforms bind to DPP IV via their carbohydrate chains and induce a Ca²⁺ signaling cascade; however, Pg 2ε alone is also able to significantly stimulate expression of MMP-9. We further demonstrate that Pg-mediated invasive activity of 1-LN cells is dependent on the availability of Pg 2ε. This is the first demonstration of a direct association between expression of MMP-9 and the Pg activation system.

Introduction

The development of an aggressive phenotype, commonly associated with the invasive behavior of many tumors, involves the increased expression of proteinases that can digest components of the extracellular matrix (ECM)¹, thus permitting passage of malignant cells through basement membranes and stromal barriers [1]. Among these enzymes, urinary-type plasminogen (Pg) activator (u-PA) and a variety of matrix-degrading metalloproteinases (MMPs) including MMP-2 and 9 play important roles [2-5]. Of particular relevance is the observation that these enzymes are secreted as inactive zymogens (prou-PA, proMMPs) which are activated extracellularly by limited proteolysis. Trace amounts of plasmin (Pm) can activate prou-PA [4], thus generating a self-maintaining feedback mechanism in which activation of prou-PA catalyzes conversion of Pg to Pm. Pg binding occurs in close proximity to the u-PA/u-PA receptor (uPAR) complex and serves to facilitate Pg activation, confine Pm to desired sites of action, and protect Pm, as well as its activator, from their respective inhibitors [4]. Pm directly activates proMMP-2 and proMMP-9 either in solution [6,7] or when both MMPs are associated with the cell surface [5,8]. ¹The abbreviations used are: ECM, extracellular matrix; MMP, matrix metalloproteinase; Pg, plasminogen; Pm, plasmin; Pg 1, plasminogen type 1; Pg 2, plasminogen type 2; DPP IV, dipeptidyl peptidase IV (CD26); u-PA, urinary-type plasminogen activator; uPAR, u-PA receptor; t-PA, tissue-type plasminogen activator; mAb, monoclonal antibody; FAPα; fibroblast activation protein α; HBSS, Hanks' balanced salt solution; Fura-2/AM, 1-[2-(5-Carboxyoxazol-2-oxyl)-6-aminobenzofuran-5-oxyl]-2-(2′-amino-5′-methylpheno-xyethane) N,N,N′,N′-tetraacetic acid acetoxy-methyl ester; 6-AHA, 6-aminohexanoic acid; L-lac, L-lactose; APMA, p-aminophenylmercuric acetate; SDS, sodium dodecyl sulfate; PAGE, polyacrylamide gel electrophoresis; FACS, fluorescence-activated cell sorter; RT-PCR, reverse transcriptase-polymerase chain reaction.

The regulation of expression and activity of MMP-9 is more complex than that of most other MMPs [9]. MMP-9 is not produced constitutively by most cells [10,11], but its activity is induced by different stimuli depending on the cell type [12,13], thereby providing a means of increasing its activity in response to specific pathophysiological events. For instance, MMP-9 is expressed at high levels by human prostate cancer, but is absent in normal prostatic tissue [14,15]. Highly invasive DU-145, PC-3, and 1-LN human prostate tumor cell lines synthesize and secrete large amounts of u-PA and proMMP-9 [16,17].

In human rheumatoid synovial fibroblasts, cell binding of Pg and its activation by u-PA induces a significant rise in cytosolic free Ca²⁺, [Ca²⁺]ᵢ, [18], via interaction of Pg/Pm with the integrin α_{IIb}β₃ and dipeptidyl peptidase IV (DPP (IV) on the cell surface [19,20]. DPP IV activities are also elevated in malignant human prostate cancers [21]. DU-145 and PC-3 cells express the integrin α_{IIb}β₃ on their surface [22]; however, expression of this integrin or DPP IV by 1-LN cells has not been assessed. Since expression of MMP-2 by human melanoma, fibrosarcoma, and ovarian cancer cells is regulated by receptor-dependent Ca²⁺ influxes [23], we investigated the possibility that a similar regulatory signal transduction mechanism participates in MMP-9 production by 1-LN cells. Pg type 2 (Pg 2) has six glycoforms that differ in their sialic acid content [24]. Extensive research has demonstrated that sialic acid content affects not only the activation of Pg, but also its function [24-27]. In the current investigation, we studied the function of single Pg 2 glycoforms after binding to 1-LN human prostate cancer cells and found that Pg 2α and Pg 2β bind to an L-lysine site-dependent receptor, whereas the highly sialylated Pg 2γ, Pg 2δ, and Pg 2ε glycoforms bind primarily to DPP IV. We also present data suggesting that DPP IV in association with Pg 2ε alone regulates expression of proMMP-9.

Experimental Procedures

Materials—Culture media were purchased from Life Technologies Inc. (Gaithersburg, Md.). 1-[2-(5-carboxyoxazol-2-oxyl-6-aminobenzofuran-5-oxyl]-2-(2′-amino-5′-methylphenoxyethane-N,N,N′,N′-tetracetic acid)-acetoxymethyl ester (Fura-2/AM) was obtained from Molecular Probes, Inc. (Eugene, Oreg.). Two-chain, high molecular weight u-PA (Mᵣ˜54,000) was obtained from Calbiochem (Richmond, Calif.). The chromogenic Pm substrate Val-Leu-Lys-p-nitroanilide (VLK-pNA, S-2251) and the chromogenic DPP IV substrate Gly-Pro-p-nitroanilide were purchased from Sigma Chemical Co. (St. Louis, Mo.). Other reagents used were of the highest grade available.

Antibodies—The monoclonal antibody (mAb) SZ21 (IMMUNOTECH, Inc., Westbrook, Me.) binds specifically to the platelet GPIIIa (β₃)-subunit [28]. Anti-dipeptidyl peptidase IV mAb clone 236.3 [29] was a generous gift of Dr. Douglas C. Hixson (Brown University, Providence, R.I.). Anti-u-PA mAb 390, and goat anti-human recombinant tissue-type Pg activator (t-PA) IgG, both anti-catalytic, were purchased from American Diagnostica (Greenwich, Conn.). Anti-fibroblast activation protein a (FAP α), mAb F19 [27], was a gift of Dr. Pilar Garin-Chesa (Thomae GmbH, Biberach, Germany). The anti-catalytic anti-MMP-9 mAb, clone 6-6B [28], was purchased from Oncogene Research Products (Cambridge, Mass.). Goat anti-mouse IgG-alkaline phosphatase conjugate antibodies were purchased from Sigma Chemical Co.

Proteins—Pg was purified from human plasma by affinity chromatography on L-lysine-Sepharose [32] and separated into its two classes of isoforms, types 1 and 2, by affinity chromatography on concanavalin A-Sepharose [33]. Fractionation of Pg 2 into its 6 glycoforms and measurement of sialic acid content were performed as previously described [24]. The mean distribution of the first five Pg 2 glycoforms in native Pg 2 was calculated from the yields obtained for each purified glycoform using chromatofocusing on a Mono P column linked to an FPLC system [24] from five separate preparations. The proportion of Pg 2φ was calculated from the amount of protein obtained after chromatography of native Pg 2 on a Sambucus nigra agglutinin lectin-Sepharose column [34], and also represents the mean value of five separate preparations. Radioiodination was carried out by the method of Markwell [35]. Radioactivity was measured in a Pharmacia LKB Biotechnology 1272 gamma counter (Rockville, Md.). Incorporation of ¹²⁵I was ˜8×10⁶ cpm/nmol of protein. ¹²⁵I-labeled Pg was repurified by affinity chromatography on L-Lysine-Sepharose and then used for the binding experiments.

Cell Cultures—The human prostate tumor cell line 1-LN was grown in RPMI 1640 supplemented with 10% fetal bovine serum, 100 units/ml penicillin G, and 100 ng/ml streptomycin.

Purification of DPP IV from 1-LN Cell Membranes—Cells grown in 20 culture flasks (150 cm²) were detached with 10 mM EDTA in Hanks' balanced salt solution (HBSS) and pelleted by centrifugation. The cell pellet was suspended in 10 ml of 20 mM Hepes, pH 7.2, containing 0.25 M sucrose and 0.5 mg/ml each of the following proteinase inhibitors: antipain-HCl, bestatin, chymostatin, transepoxysuccinyl-L-leucylamido-(4-guanidino)butane (E-64), leupeptin, pepstatin, O-phenanthroline, and aprotinin. Cells were lyzed by sonication on ice (five 10 s bursts with 30 s intervals). All procedures were performed at 4° C. The homogenate was centrifuged at 800×g for 15 min to remove unbroken cells and nuclei, followed by centrifugation of the supernatant at 50,000×g for 1 h. The pellet containing cell membranes was resuspended in 20 mM Tris-HCl, pH 8.0, containing 1% (v/v) Triton X-100 to solubilize membranes and centrifuged again at 50,000×g for 30 min to remove insoluble materials. DPP IV activity in this supernatant and in all the following purification steps was monitored by a chromogenic assay using the DPP IV substrate Gly-Pro-pNA [36]. The enzyme was sequentially purified to homogeneity using DEAE-Sepharose ion exchange chromatography and Gly-Leu-Sepharose affinity chromatography [37], followed by chromatography on concanavalin A-Sepharose and gel filtration on a Sepharose S-200 column. These steps yielded fully active DPP IV (˜40 μg/1×10⁹ cells). Electrophoretic analysis showed an essentially homogenous protein. A sample of the protein was analyzed by matrix-assisted laser desorption ionization-MS, and the obtained mass spectrometric peptide maps (30 peptides) were used to identify DPP IV in the OWL Protein database release 29.6 [38,39].

Protein Sequence Analysis—The proteins (100 pmol) were sequenced by automatic Edman degradation in a gas/liquid phase sequencer (model 477A; Applied Biosystems, Inc., Foster City, Calif.) with online PTH analysis using HPLC (model 120A; Applied Biosystems, Inc., Foster City, Calif.). The instruments were operated as recommended in the user bulletins and manuals distributed by the manufacturer.

Ligand Binding Analysis—Cells were grown in tissue culture plates until the monolayers were confluent. Prior to use in binding assays, the cells were washed in HBSS. All binding assays were performed at 4° C. in RPMI 1640 containing 2% bovine serum ablumin (BSA). Increasing concentrations of ¹²⁵I-labeled Pg 2 glycoforms were incubated with cells for 60 min in 48-well or 96-well culture plates, respectively. Free ligand was separated from bound by aspirating the incubation mixture by and washing the cell monolayers rapidly three times with RPMI 1640 containing 2% BSA. The cells were then lyzed with 0.1 M NaOH, and bound radioactivity was determined in a Pharmacia LKB Biotechnology 1272-gamma counter. Molecules of ligand bound were calculated after substraction of non-specific binding measured in the presence of nonlabeled 100 μM Pg 2. Estimates for dissociation constant (K_{d}) values and maximal binding of Pg 2 glycoforms (Bₘₐₓ) were determined by fitting data directly to the Langmuir isotherm using the statistical program SYStat® for Windows.

Solid Phase Radioligand Binding Studies—To study specific binding of Pg 2 glycoforms to immobilized DPP IV purified from 1-LN cells, 96-well strip plates were coated with DPP IV (1 μg/ml in 0.1 M sodium carbonate, pH 9.6, 200 μl/well, 37° C., 2 h). After coating, plates were washed with 200 μl of 10 mM sodium phosphate, 100 mM NaCl, pH 7.4, containing 0.05% Tween-80 (PBS-Tween) to remove unbound protein. Non-specific sites were blocked by incubating with PBS-Tween containing 2% BSA at room temperature for 1 h. Plates were rinsed twice with 200 μl of PBS-Tween, air dried, and stored at 4° C. For assays, increasing concentrations of ¹²⁵I-labeled Pg 2 glycoforms, with or without 50-fold excess of unlabeled ligands, were added to triplicate wells and incubated at 37° C. for 1 h. Following incubation, the supernatants were removed and the plates rinsed three times with 200 μl PBS-Tween. Wells were stripped from the plates and radioactivity measured. Specific binding was calculated by substraction of non-specific binding measured in the presence of unlabeled ligand.

Measurement of Intracellular Calcium Levels—Cystolic free calcium [Ca²⁺]ᵢ, was measured by Digital Imaging Microscopy (DIM) using the fluorescent indicator Fura-2/AM as previously described [18].

Gelatin Zymography—Protein samples were electrophoresed on gelatin-containing 0.75 mm thick 10% polyacrylamide gels in the presence of SDS under nonreducing conditions [40]. After completion of the electrophoretic run, the gels were incubated with two changes of 2.5% Triton X-100 for 1 h, followed by incubation for 18 h at 37° C. in 0.1 M glycine-NaOH, pH 8.3, containing 1 mM CaCl₂, and 0.1 M ZnCl₂, before staining with Coomassie Brilliant Blue R-250 to visualize the lysis bands.

MMP-9 Activity in Solution—MMP-9 activity was measured in tissue culture supernatants by quantitative zymography [41] using as a standard MMP-9 purified by affinity chromatography on gelatin-Sepharose from 1-LN cell conditioned medium (10 liters) [42]. Conditioned medium (50 μl), from 1-LN cell monolayers in 48 well culture plates (1.7×10⁶ cells/well) incubated with Pg 2 glycoforms and/or inhibitors of Pg binding or activation, were electrophoresed on gelatin-containing gels and the degree of lysis was quantified using a Gelman ACD-15 Automatic Computing Densitometer (Gelman Instrument Company, Ann Arbor, Mich.). Values were determined by integrating the density of the selected bands and expressed in units×mm². Each gel was scanned three times and the average value of the integrated density of the bands was used to determine levels of MMP-9 from calibration curves constructed with purified active MMP-9 electrophoresed under the same conditions. The statistical analysis of the data was performed on an IBM 433 DX/S computer using the program SYSTAT® for Windows 95. The statistical significance of differences between means was evaluated by Student's t-test. MMP-9 was positively identified in conditioned medium by electrophoretic separation in 10% SDS-polyacrylamide gels (SDS-PAGE), electroblot of the electrophoresed proteins to nitrocellulose membranes and reaction with an anti-MMP-9 mAb (1 μg/ml) followed by reaction with a secondary alkaline phosphatase conjugated anti-mouse IgG. Detection was performed by reaction with the alkaline phosphatase substrate 5-bromo-4-chloro-3-indolyl phosphate in the presence of nitroblue tetrazolium (1 mM each) in 10 mM Tris-HCl, pH 8.5.

Gel Electrophoresis—Electrophoresis was performed on polyacrylamide gels (1.2-mm thick, 14×10 cm) containing 0.1% SDS. A discontinuous Laemli buffer system was used [43]. Visualization of the proteins was carried out by staining the gel with 0.25% Coomassie Brilliant Blue R-250 in 45% methanol/10% acetic acid. Transfer to nitrocellulose paper was carried out by the Western blot method [44]. The dye-conjugated molecular weight markers (BioRad, Richmond, Calif.) used were myosin (Mᵣ=218,000), β-galactosidase (Mᵣ=134,000), bovine serum albumin (Mᵣ=84,000), carbonic anhydrase (Mᵣ=44,000) and soybean trypsin inhibitor (Mᵣ=32,000).

Flow Cytometry—1-LN cells were grown at 37° C. in RPMI 1640 containing 10% fetal bovine serum as adherent monolayers. Cells were detached by incubation for 5 min at 37° C. with Ca²⁺ and Mg²⁺-free PBS containing 10 mM EDTA and then pelletted. Cells were resuspended in ice-cold staining buffer (phenol red-free HBSS, 1% BSA, 0.1% NaN₃) at a concentration of 1×10⁷ cells/ml. Aliquots (100 μl) of these cells suspensions were incubated for 90 min on ice with an appropriate dilution of either FITC-conjugated anti-human DPP IV, FITC-conjugated anti-human GPIIIa (p3) or a FTIC-conjugated isotype control murine monoclonal antibody. For analyses of cell-surface FAPα, cells were first incubated on ice with the anti-FAPα mAb F19 for 90 min, and then for an additional 90 min with a FITC-conjugated anti-mouse IgG. Cells were then rinsed three times with ice-cold staining buffer, resuspended in ice cold 10% formalin, and stored in the dark at 4° C. until analyses by flow cytometry. The mean relative fluorescence after excitation at a wavelength of 408 nm was determined for each sample on a FACScan flow cytometer (Becton-Dickinson, Franklin Lakes, N.J.) and analyzed with CELLQUEST™ software (Becton-Dickinson, Franklin Lanes, N.J.).

RNA Isolation—To determine changes in MMP-9 mRNA induced by Pg, 1-LN cells were grown in 48 well culture plates (1.7×10⁶ cells/well) and incubated with each individual Pg 2 glycoform for 24 h at 37° C. Cell monolayers were then rinsed twice in serum-free RPMI 1640 and total RNA extracted by a single-step method, using RNeach Mini kit (Qiagen, Chatsworth, Calif.), according to the manufacturer's instructions.

Measurement of MMP-9 mRNA Levels by Reverse Transcription-PCR (RT-PCR)—Total RNA was reverse transcribed with 1 μg of RNA in a 20 μl reaction mixture, using M-MLV reverse transcriptase (200 U) and oligo d(T) as primer for 1 h at 42° C. The resulting cDNA (5 μl) was used as a template and a 212-bp segment of the MMP-9 cDNA was amplified, using a 24-mer upstream primer (5′-AGTTGAACCAGGTGGACCAAGTGG-3′), identical to positions 2079-2102 and a 29-mer downstream primer (5′-AACAAAAAACAAAGGTGAGAAGAGAGGGC-3′) complimentary to positions 2270-2298 of the human MMP-9 mRNA [45]. A 600-bp segment of the glyceraldehyde phosphate dehydrogenase (GAPDH, constitutive internal control) cDNA was co-amplified, using a 24-mer upstream primer (5′-CCACCCATGGCAAATTCCATGGCA-2′), identical to positions 212-235 and a 24-mer downstream primer (5′-TCTAGACGGCAGGTCAGGTCCACC-3′), complimentary to positions 786-809 of the human GAPDH mRNA [46]. Amplification was carried out in a Techne Thermal Cycler PHC-3 for 28 cycles (one cycle=94° C. for 45 s, 60° C. for 45 s, and 72° C. for 45 s). PCR products were analyzed on a 1.2% agarose-ethidium bromide gel. The gels were photographed and the intensity of the individual MMP-9 and GAPDH mRNA bands measured by laser densitometric scanning, using a Molecular Dynamics Personal Densitometer. Changes in MMP-9 mRNA levels were expressed as a relative ratio of MMP-9 mRNA/GAPDH mRNA band intensities.

In Vitro Invasion Assay—The invasive activity in vitro was assessed by determining the ability of 1-LN cells to invade Matrigel® [47]. Polycarbonate filters (8-μm pore size; Becton Dickinson, Franklin Lakes, N.J.) were coated with Matrigel (12 μg/filter) and placed in a modified Boyden chamber. Cells (1×10⁵) were added to the upper chamber in serum-free RPMI 1640 medium, or medium containing purified Pg 2 glycoforms in the absence and presence of anti-DPP IV, anti-u-PA or anti-MMP-9 IgGs, and incubated for 48 h in a humidified atmosphere. Following incubation, non-invading cells were removed from the upper chamber with a cotton swab, and filters were excised and stained with Cyto-Quik™ (Fisher Scientific, Fair Lawn, N.J.). Cells on the lower surface of the filter were enumerated using an ocular micrometer and counting a minimum of five high-powered fields. Each experiment was performed twice with triplicate samples.

Results

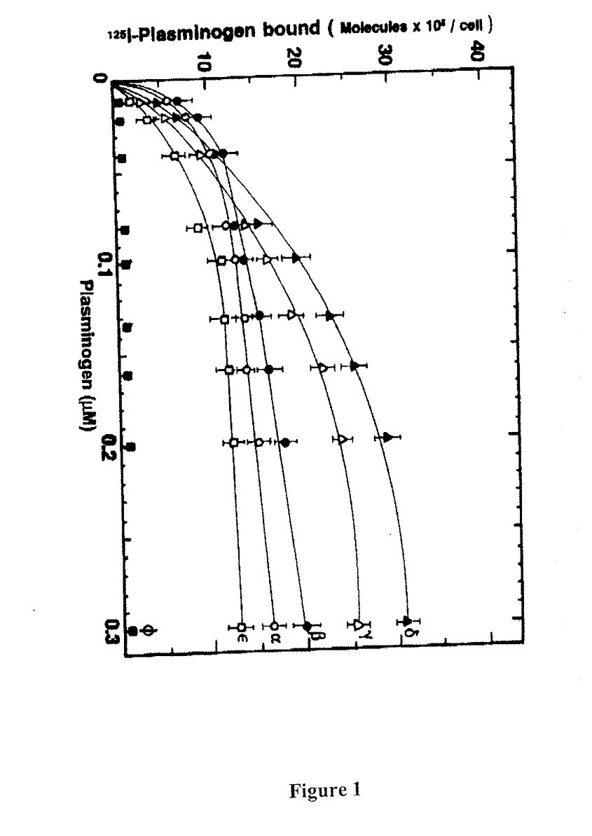

Binding of Single Pg 2 Glycoforms to 1-LN Human Prostate Tumor Cells—Binding of ¹²⁵I-labeled single Pg 2 glycoforms to 1-LN cells was determined as described under Experimental Procedures. Native Pg 2 has six glycoforms which differ in their sialic acid content [24]. Binding experiments (FIG. 1) show that Pgs 2α, β, γ, δ, and ε (1.3, 2.2, 2.95, 5.77 and 5.34 mol sialic acid/mol Pg, respectively) bind to 1-LN cells in a dose-dependent manner with high affinity and to a large number of sites (Table I). Pg 2φ (13.65 mol sialic acid/mol Pg) does not bind to 1-LN cells.

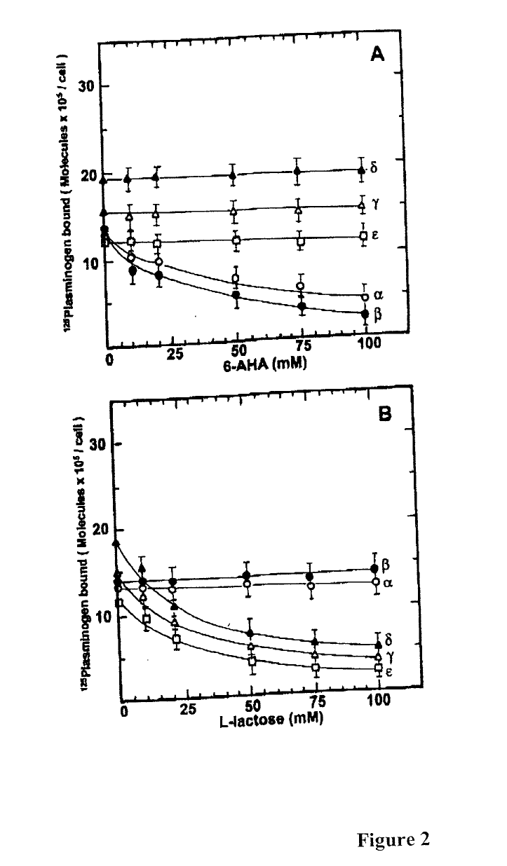

In order to assess the binding mechanism of the individual Pg 2 glycoforms to 1-LN cells, we studied their activation by cells incubated with each glycoform in the presence of 6-aminohexanoic acid (6-AHA) and L-lactose. The antifibrinolytic amino acid 6-AHA prevents interaction of Pg L-lysine binding sites with several cell membrane-associated components [48,49]. L-lactose is a sugar which intereferes with binding of Neu 5-AC (α2-3) or (α2-6) residues to sialic acid binding proteins [50] and inhibits binding of Native Pg 2 to DPP IV on the surface of rheumatoid synovial fibroblasts [20]. Incubation of the cells with single Pg 2 glycoforms in the presence of increasing concentrations of 6-AHA inhibited the binding of Pg 2α and Pg 2β (FIG. 2A), whereas increasing concentration of L-lactose inhibited binding of Pgs 2γ, 2δ, and 2ε (FIG. 2B). Taken together, these experiments suggest that Pgs 2α and 2β bind to 1-LN cells via their L-lysine binding sites, and Pgs 2γ, δ, and ε bind via their carbohydrate chains. The activation of Pg 2 glycoforms is inhibited by anti-u-PA antibodies and is not affected by anti-t-PA antibodies, suggesting that u-PA is the primary Pg activator at the surface of 1-LN cells (data not shown).

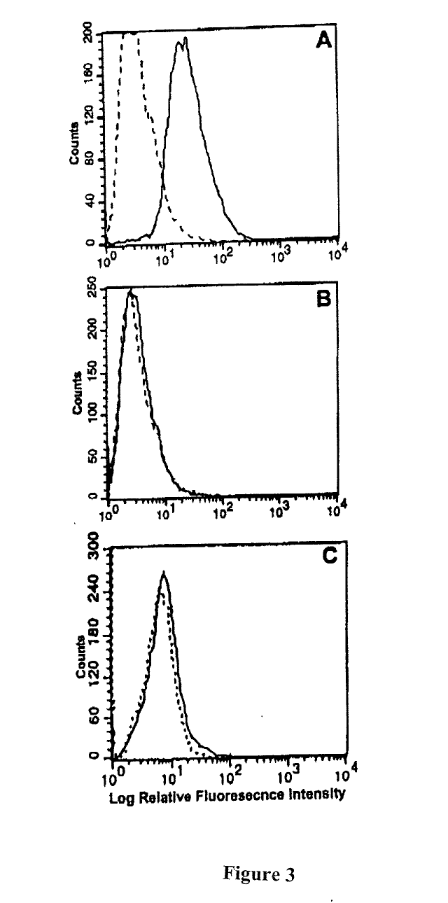

Analyses of Binding of DPP-IV, β₃, and FAPα Antibodies to the Surface of 1-LN Cells by Flow Cytometry—1-LN cells were analyzed by fluorescence-assisted flow cytometry (FACS) as described under Experimental Procedures. The mAbs SZ21 specific for the platelet GPIIIa (β₃) antigen and clone 236.3 specific for human DPP IV were used for these experiments. As determined by FACS of 1-LN cells reacted with FITC-labeled IgGs, cells react with the anti-DPP IV antibody (FIG. 3A), whereas the cells show no detectable GPIIIa (β₃) antigen on their surface (FIG. 3B). In rheumatoid synovial fibroblasts, the integrin β₃ serves as a L-lysine binding site receptor for Pg, whereas DPP IV is a Pg sialic acid receptor [19,20]. The absence of β₃ in 1-LN cells suggests a different L-lysine binding site for Pg in these cells.

DPP IV shares 48% amino acid sequence identity with the human fibroblast activation protein a (FAPα) [51], a cell surface antigen selectively expressed in reactive stromal fibroblasts of epithelial cancers and malignant bone and soft tissue sarcoma cells [52]. Since DPP IV and FAPα share the amino acid sequence LQWLRR [51], which in rheumatoid synovial fibroblast DPP IV serves as the binding site for Pg carbohydrate chains [20], we investigated the expression of FAPα on the surface of 1-LN cells. We used the mAb F19 which is specific for FAPα, but non cross-reactive with DPP IV [52,53]. 1-LN cells reacted with mAb F19 and then analyzed by FACS showed no detectable FAPα on their surface (FIG. 3C).

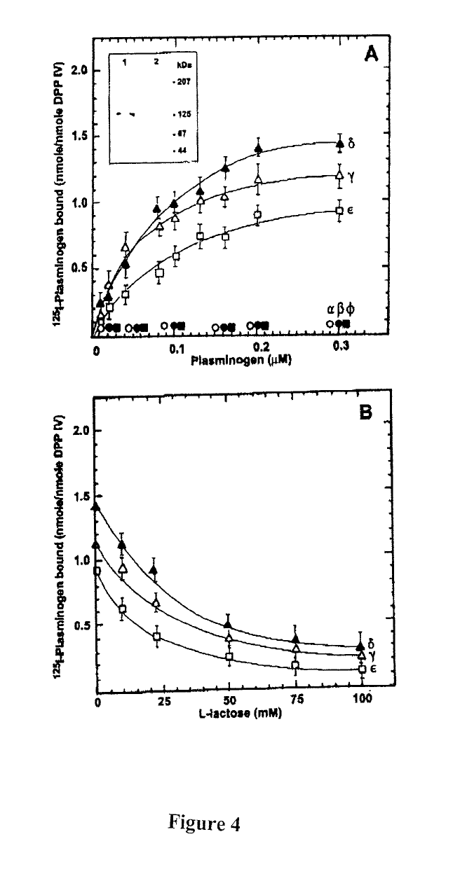

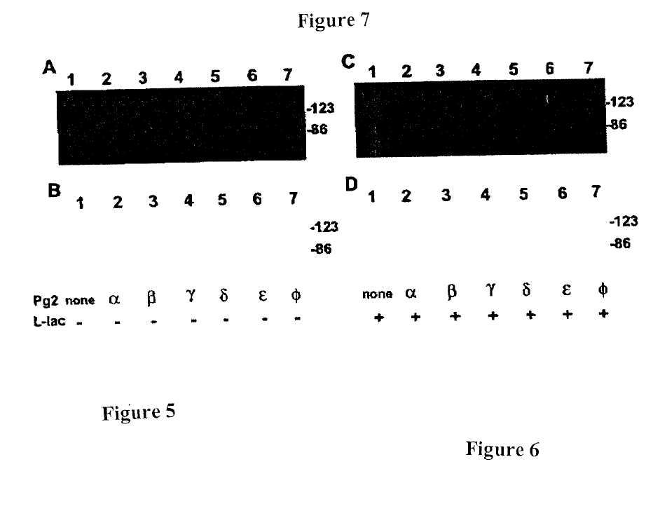

Binding of Pg 2 Glycoforms to Immobilized DPP IV Isolated from 1-LN Cell Membranes—Once identified as a Pg receptor, DPP IV from 1-LN cell membranes was purified to homogeneity as described under Experimental Procedures. Electrophoretic analysis of the protein is shown in FIG. 4A. A Coomassie Brilliant Blue R-250 stain of the electrophoresed material (FIG. 4A, Inset: lane 1) shows a major protein band in the Mᵣ˜120,000 size range. A blot binding assay with mAb clone in the Mᵣ˜120,000 size range. A blot binding assay with mAb clone 236.3 specific for DPP IV [26] shows reactivity only with the Mᵣ˜120,000 protein band (FIG. 4A, Inset: lane 2). DPP IV immobilization on cell culture plates and binding assays of Pg 2 glycoforms were performed as described under Experimental Procedures. Only Pgs 2γ, δ, and ε bind to this DPP IV in a dose-dependent and saturable manner (FIG. 4A). No specific binding was observed with Pgs 2α, β, and φ. Binding of each individual ¹²⁵I-labeled Pg 2γ, 2δ, and 2ε (0.1 μM each) to DPP IV in the presence of increasing concentrations of L-lactose is progressively inhibited (FIG. 4B), suggesting that Pg sialic acid residues are involved in this interaction. Since Pgs 2γ, δ, and ε represent over 65% of the distribution of Pg 2 glycoforms and Pg 2φ is unable to bind (Table I), these results suggest that DPP IV is the primary Pg 2 receptor in 1-LN cells.

[Ca²⁺]ᵢ Response to Pg 2 Glycoforms Binding on the Surface of 1-LN Cells—We measured changes in [Ca²⁺]ᵢ after binding of each individual Pg 2 glycoform to the surface of 1-LN cells. Pgs 2α and β did not produce any changes (FIGS. 5A and 5B, respectively). However, binding of Pgs 2γ, δ, or ε elicited a [Ca²⁺]ᵢ response (FIGS. 5C, 5D, and 5E, respectively). No response was observed with Pg 2φ (FIG. 5F). Similarly, cells were incubated at 37° C. for 1 h with anti-u-PA or anti-p IgGs (100 μg/ml) which inhibit enzymatic activity prior to addition of the highly sialylated glycoforms (Pgs 2γ, 2δ, and 2ε). Neither cell population demonstrated major changes in their [Ca²⁺]ᵢ responses (data not shown). However, L-lactose (100 mM) which prevents the interaction of Pg carbohydrate chains with DPP IV [24] was able to inhibit the response induced by Pgs 2γ, δ or ε (FIGS. 5G, 5H and 5I, respectively). A similar inhibition of the (Ca²⁺)ᵢ response (data not shown) was observed when the cells were pre-incubated with the anti-DPP IV mAb 236.3 (50 μg/ml) before addition of these glycoforms. These results are consistent with the observations reported above, suggesting that the [Ca²⁺]ᵢ response is the result of a direct interaction between the highly sialylated glycoforms (Pgs γ, δ, and ε) and DPP IV on the cell surface, and does not require Pg activation.

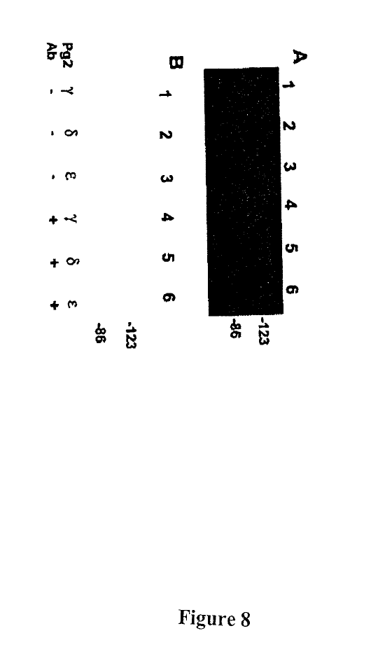

Effect of Pg on the Expression of MMP-9 by 1-LN Cells—Cells were seeded into 48-well culture plates and grown in RPMI 1640 containing 10% fetal bovine serum. Confluent monolayers were then incubated for 24 h with quiescent culture medium containing RPMI 1640 and 0.5% fetal bovine serum. Each individual Pg 2 glycoform (0.1 μM) was added in triplicate to cell monolayers in 300 μl of serum-free RPMI 1640 and incubated for 24 h at 37° C. Culture medium was collected to measure secretion of MMP-9 as described under Experimental Procedures. Prior to analyses of the MMP-9 secreted into the medium by 1-LN cells in the presence of individual Pg 2 glycoforms, we purified MMP-9 from conditioned medium (5 liters) by the technique of Masure et al. [42]. Analyses of the purified MMP-9 are shown in FIG. 6. An electrophoretic analysis of the purified protein shows a major band with Mᵣ˜85,000 and a minor band with Mᵣ˜95,000 proteins (FIG. 6, lane 1). An electroblot analysis with an anti-MMP-9 mAb shows reaction of the antibody with both the Mᵣ˜85,000 and 95,000 proteins (FIG. 6, lane 2). Gelatin zymography of the proteins shows activity only in association with the Mᵣ˜85,000 protein (FIG. 6, lane 3). Amino-terminal sequence analysis demonstrated the sequence FQTFEGDL, [42] corresponding to the amino-terminal sequence of active MMP-9. A similar analysis of the Mᵣ˜95,000 protein yielded the sequence APRQRQ, corresponding to the amino-terminal sequence of proMMP-9. These results suggest that most of the MMP-9 secreted into the culture medium by 1-LN cells is in the active form. We then proceeded to analyze the MMP-9 secreted into the medium by 1-LN cells incubated with each individual Pg 2 glycoform in serum-free culture medium, using the purified MMP-9 as a standard for quantification by gelatin zymography. These analyses (FIG. 7A) show a major band of active protein with a Mᵣ˜85,000. Quantification of this protein (Table II) demonstrates a 3-fold stimulation of active MMP-9 secreted by 1-LN cells incubated with Pg 2ε when compared to cells incubated with other Pg 2 glycoforms or culture medium (p<0.001). Samples of these conditioned media were also subjected to SDS-PAGE under reducing conditions, electroblotted to nitrocellulose membranes and then reacted with an anti-MMP-9 mAb (FIG. 7B). These studies also suggest that only Pg 2ε stimulates production of MMP-9. Cells co-incubated with 6-AHA (100 mM) and individual Pg 2 glycoforms did not show any major changes in the production of MMP-9 when compared with controls (Table II). A zymogram of conditioned media from cells incubated with each individual Pg 2 glycoform in the presence of L-lactose (100 mM) (FIG. 7C) shows an average decrease in the production of MMP-9 for every Pg 2 glycoform, with the exception of Pg 2ε which shows a 12-fold decrease in the production of MMP-9 (p<0.0001) (Table II), at levels almost undetectable in an electroblot reacted with an anti-MMP-9 mAb (FIG. 7D). A 4-fold decrease in the production of MMP-9 by cells co-incubated with anti-DPP IV mAb 236.3 and Pg 2ε (p<0.001) (Table II) is clearly observed in the conditioned medium (Lane 6 on FIGS. 8A and 8B, respectively).

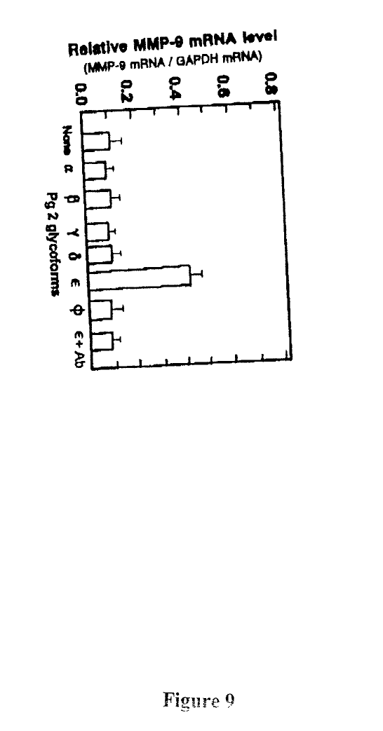

Measurements of the relative changes in MMP-9 mRNA levels (FIG. 9) show a significant increase in expression of MMP-9 mRNA in cells incubated with Pg 2ε. Cells incubated with Pg 2α, 2β, 2γ, or 2δ glycoforms, however, did not show a significant change in their relative mRNA levels (ratio MMP-9 mRNA/GAPDH mRNA) when compared with control cells incubated with serum-free medium alone. Cells co-incubated with Pg 2ε and a binding inhibitory anti-DPP IV IgG did not show a change in the relative MMP-9 mRNA levels when compared with control cells incubated with serum-free medium alone. Taken together, these results suggest that Pg 2ε not only significantly stimulates expression of MMP-9, but it is also involved in its activation.

Effect of Pg on 1-LN Cellular Invasion—Pg enhances the ability of prostate cancer PC-3 and DU-145 cell lines to penetrate the synthetic basement membrane Matrigel® [54]. To determine whether Pg regulates invasion via secretion of MMP-9, 1-LN cells were incubated with different inhibiting antibodies in the presence of purified Pg 2 glycoforms. Table III shows that Pg 2ε enhances cellular invasion 6-7-fold. Co-incubation of Pg 2ε with anti-u-PA or anti-MMP-9 which inhibit enzymatic activity reduces invasiveness to nearly undetectable levels. Similar results are observed with cells co-incubated with Pg 2ε and anti-DPP IV IgG. These results further demonstrate that Pg 2ε is the only glycoform that significantly enhances 1-LN cell invasive activity, an effect resulting from its capacity to stimulate expression of MMP-9.

Discussion

Degradation of ECM components occurs during a variety of tissue remodeling processes, including tumor invasion and rheumatoid arthritis. A complex mechanism requiring the fibrinolytic system and MMPs governs tumor stromal generation and development of a vascular pannus in rheumatoid arthritis [55]. In both abnormalities, the Pg activation system and production of MMPs are upregulated, leading to the degradation of ECM components which contribute to both articular destruction in rhematoid arthritis and penetration of basement membranes by spreading cancer cells [55,56]. For these reasons, we investigated the possibility that similar Pg receptors also existed in human prostate cancer cells and that they are involved in regulation of MMP-9 expression and activation. We studied the highly invasive 1-LN human prostate tumor cell line [57] because these cells synthesize and secrete large amounts of u-PA and MMPs [17]. Unlike rheumatoid synovial fibroblasts, we did not find the β₃ integrin associated with the membrane glycoprotein DPP IV. Our findings are summarized in Table IV. The less sialylated Pg 2α and Pg 2β bind to these cells via a L-lysine binding sites, they do not elicit a [Ca²⁺]ᵢ response, and they are not involved in secretion or expression of MMP-9. Pg 2γ, Pg 2δ, and Pg 2ε bind to DPP IV via their sialic acid residues and induce a [Ca²⁺]ᵢ response [20]; however, only Pg 2ε is able to induce expression and secretion of MMP-9. The [Ca²⁺]ᵢ response in synovial fibroblasts requires binding of Pg to the integrin β₃ and activation by u-PA before their interaction with DPP IV [19,20], whereas in 1-LN cells a direct reaction of Pg with DPP IV induces a similar response. The identity of the L-lysine dependent receptors of Pgs 2α and 2β on 1-LN cells remains unknown; however, due to its potential as a regulatory site, we are currently investigating its identity.

In the circulation, the concentration of Pg 2 is 2-fold greater than Pg 1; however, in the extravascular space the concentration of Pg 2 is almost 6-fold greater than Pg 1 [58]. Pg 1 contains one O-glycan at Thr-345 and one biantennary N-glycan at Asn-288, whereas Pg 2 contains only the O-glycan chain [59,60]. Pg 1 activation is enhanced more than that of Pg 2 in the presence of fibrin by either u-PA or t-PA [61], suggesting a preferred role for Pg 1 in the intravascular space [58]. The shift in the ratio of Pg 2 to Pg 1 in the extravascular space suggests a significant role for Pg 2 glycoforms as the preferred forms for Pm formation during metabolism on the cell surface [62]. In this context, Pg 2ε should preferentially function at the cell surface, where its carbohydrate content, in general, and sialic acid, in particular, may play an important role in regulating its function.

Pgs 2δ and 2ε contain almost the same amount of sialic acid (5.77 and 5.34 mol sialic acid/mol Pg). However, the pI of Pg 2ε is more acidic [24], suggesting an additional secondary modification of its structure which may be critical for its capacity to induce expression of MMP-9. This shift in the pI of Pg 2ε may be associated with phosphorylation of the Pg molecule [63]. In this context, a shift in the pI of u-PA from 9.2 to 7.6 secondary to Tyr and Ser phosphorylation is associated with the activation of pp60src and of protein kinase C in metastatic tumor cells [64-66]. However, no data are available with respect to the kinases involved in Pg and u-PA phosphorylation or its role in the multiple biological functions exerted by these proteins.

Pg-mediated invasive activity of 1-LN cells is effectively blocked by mAbs which inhibit the enzymatic activity of u-PA or MMP-9, suggesting that Pm in the tumor cell micro-environment can enhance the invasive activity either by direct proteolysis of ECM components [13,67] or via its capacity to activate proMMP-9 bound to a cell surface [5]. Recent studies in mice with targeted inactivation of the t-PA, u-PA or Pg genes [68], suggest that proMMP-9 activation may occur in the absence of t-PA or u-PA, whereas no active MMP-9 is detected in the absence of Pg. The mechanism whereby Pg is activated in this setting is unknown. Our studies demonstrate that Pg influences cell migration not only by its capacity to generate Pm which degrades fibrin, but also because it stimulates MMP-9 expression and activation.

In addition to the multiple functions that DPP IV performs on T cells, where it is known as CD26 [69], this glycoprotein is an endothelial cell adhesion molecule mediating lung metastases by rat breast cancer cells [70]. Expression of MMP-2 and MMP-9 by A2058 human melanoma cancer cells are also independently regulated by receptor-operated Ca²⁺ influxes, although no specific physiological ligand has been identified [23,71,72]. Our results provide new evidence connecting DPP IV with the Pg activation enzymatic system and expression of MMP-9, and suggest a biochemical mechanism by which Pg might regulate MMP-9 in the extracellular environment.

### REFERENCES

1. Nagase, H. (1996) Matrix metalloproteinases. In: Zinc Metalloproteinases in Health and Disease, (Hooper, N.M., ed.), Taylor and Francis, London, England, pp. 153-204

2. DeClerk, Y. A. and Laug, W. E. (1996) Cooperation between matrix metalloproteinases and the plasminogen activator-plasmin system in tumor progression. Enz. Prot. 49, 72-84

3. DeVries, T. J., Van Muijen, G. N. P. and Ruiter, D. J. (1996) The plasminogen activator system in tumor invasion and metastasis. Path. Res. Pract. 192, 718-733

4. Andreansen, P. A., KjØller, L., Christenson, L., and Duffy, M. (1997) The urokinase-type plasminogen activator system in cancer metastasis: a review. Int. J. Cancer 72, 1-22

5. Mazzieri, R., Masiero, L., Zanetta, L., Monea, S., Onisto, M., Garbisa, S., and Mignatti, P. (1997) Control of type IV collagenase activity by components of the urokinase-plasmin system: a regulatory mechanism with cell-bound reactants. The EMBO J. 16, 2319-2332.

6. Liotta, L. A., Goldbarb, R. H., Brundage, R., Siegal, G. P., Terranova, V., and Garbissa, S. (1981) Effect of plasminogen activator (urokinase), plasmin, and thrombin on glycoproteins and collagenous components of basement membranes. Cancer Res. 41, 4629-4636.

7. Keski-Oja, J., Lohi, J., Tuuttila, A., Tryggvason, K., and Vartio, T. (1992) Proteolytic processing of the 72,000-Da type IV collagenase by urokinase plasminogen activator. Exp. Cell. Res. 202, 471-476.

8. Makowski, G. S., and Ramsby, M. L. (1998) Amorphous calcium phosphate-mediated binding of matrix metalloproteinase-9 to fibrin is inhibited by pyrophosphate and biphosphonate. Inflammation 22, 287-305.

9. Dubois, B., Openakker, G., and Carton, H. (1999) Gelatinase B in multiple sclerosis and experimental autoimmune encephalomyelitis. Acta Neurol. Belg. 99,53-56.

10. Collier, I. E., Wilhelm, S. M., Eisen, A. Z., Marner, B. L., Grant, G. A., Seltzer, J. L., Kronberger, A., He, C., Bauer, E. A., and Goldberg, G. I. (1988) H-ras oncogene-transformed human bronchial epithelial cells (TBE-1) secrete a single metalloprotease capable of degrading basement membrane collagen. J. Biol. Chem. 263, 6579-6587.

11. Hipps, D. S., Hembry, R. M., Docherty, A. J. P., Reynolds, J. J., and Murphy, G. (1991) Purification and characterization of human 72 kDa gelatinase (typeIV) collagenase. Use of immunolocalization to demonstrate the non-coordinate regulation of the 72 kDa and 95 kDa gelatinases by human fibroblasts. Biol. Chem. Hoppe-Seyler 372, 287-296.

12. Devarajan, P., Johnston, J. J., Ginsberg, S. S., Van Wart, H. E., and Berliner, H. (1992) Structure and expression of neutrophil gelatinase cDNA. Identity with type IV collagenase from HT1080 cells. J. Biol. Chem. 267, 25228-25232.

13. Houde, M., DeBruyne, G., Bracke, M., Ingelman-Sundberg, M., Skoglund, G., Masure, S., Van Damme, J., Opdenaker, G. (1993) Differential regulation of gelatinase B and tissue-type plasminogen activator expression in human Bowes melanoma cells. Int. J. Cancer 53, 395-400.

14. Stearns, M. E., and Wang, M. (1993) Type IV collagenase (Mr 72,000) expression in human prostate: benign and malignant tissue. Cancer Res. 53, 878-883.

15. Hamdy, F. C., Fadlon, E. J., Cottam, D., Lawry, J., Thurrell, W., Silcocks, P. B., Anderson, J. B., Williams, J. L., and Rees, R. C. (1994) Matrix metalloproteinase-9 expression in primary human prostatic adenocarcinoma and benign prostatic hyperplasia. Br. J. Cancer 69, 177-182.

16. Hoosein, N. M., Boyd, D. D., Hollas, W. J., Mazar, A., Henkin, J. and Chung, L. W. K. (1991) Involvement of urokinase and its receptor in the invasiveness of human prostatic carcinoma cell lines. Cancer Comm. 3, 255-264.

17. Wilson, M. J., and Sinha, A. A. (1993) Plasminogen activator and metalloprotease activities of DU-145, PC-3, and 1-LN-PC3-1A human prostate tumors grown in nude mice: correlation with tumor invasive behavior. Cell. Mol. Biol. Res. 39, 751-760.

18. Gonzalez-Gronow, M., Gawdi, G. and Pizzo, S. V. (1993) Plasminogen activation stimulates an increase in intracellular calcium in human synovial fibroblasts. J. Biol. Chem. 268, 20791-20794.

19. Gonzalez-Gronow, Gawdi, G., and Pizzo, S. V. (1994) Characterization of the plasminogen receptors of normal and rheumatoid arthritis synovial fibroblasts. J. Biol. Chem. 269, 4360-4366.

20. Gonzalez-Gronow, M., Weber, M. R., Shearin, T. V., Gawdi, G., Pirie-Shepherd, S. R., and Pizzo, S. V. (1998) Plasmin(ogen) carbohydrate chains mediate binding to dipeptidyl peptidase IV (CD26) in rheumatoid arthritis human synovial fibroblasts. Fibrinol. Proteol. 12, 366-374.

21. Wilson, M. J., Ruhland, A. R., Quast, B. J., Reddy, P. K., Ewing, S. L., and Sinha, A. A. (2000) Dipeptidyl peptidase IV activities are elevated in prostate cancers and adjacent benign hyperlastic glands. J. Androl. 21, 220-226.

22. Trikha, M., Raso, E., Cai, Y., Fazakas, Z., Paku, S., Porter, A. T., Timar, J., and Honn, K. V. (1998) Role of the α_{IIb}β₃ integrin in prostate cancer metastasis. The Prostate 35, 185-192.

23. Kohn, E. C., Jacobs, W., Kim, Y. S., Alessandro, R., Stetler-Stevenson, W. G., and Liotta, L. A. (1994) Calcium influx modulates expression of matrix metalloproteinase-2 (72 kDa type IV collagenase, gelatinase A). J. Biol. Chem. 269, 21505-21511.

24. Pirie-Shepherd, S. R., Jett, E. A., Andon, N. L., and Pizzo, S. V. (1995) Sialic acid content of plasminogen 2 glycoforms as regulators of fibrinolytic activity. J. Biol. Chem. 270, 5877-5881.

25. Pirie-Shepherd, S. R., Serrano, R. L., Andon, N. L., Gonzalez-Gronow, M., and Pizzo, S. V. (1996) The role of carbohydrate in the activation of plasminogen 2 glycoforms by streptokinase. Fibrinolysis 10, 49-51.

26. Mori, K., Dwek, R. A., Downing, A. K., Opdenakker, G., and Rudd, P. M. (1995) The activation of type 1 and type 2 plasminogen by type I and type II tissue plasminogen activator. J. Biol. Chem. 270, 3261-3267.

27. Davidson D. J., and Castellino, F. J. (1993) The influence of the nature of the asparagine 289-linked oligosaccharide on the activation by urokinase and lysine binding properties of natural and recombinant human plasminogens. J. Clin. Invest. 92, 249-254.

28. Ruan, C., Du, X., Xi, X., Castaldi, P. A., and Berndt, M. C. (1987) A murine antiglycoprotein Ib complex monoclonal antibody, SZ2, inhibits platelet aggregation by both ristocetin and collagen. Blood 69, 570-577.

29. Walborg, E. F., Tsuchida, S., Weeden, D. S., Thomas, M. W., Barrick, A., McEntire, K. D., Allison, J. P., and Hixson, D. C. (1985) Identification of dipeptidyl peptidase IV as a protein shared by the plasma membrane of hepatocytes and liver biomatrix. Exp. Cell Res. 158, 509-518.

30. Garin-Chesa, P., Old, L. J., and Rettig, W. J. (1990) Cell surface glycoprotein of reactive stromal fibroblasts as a potential antibody target in human epithelial cancers. Proc. Natl. Acad. Sci. USA 87, 7235-7239.

31. Zucker, S., Lysik, R. M., Zarrabi, M. H., and Mol., U. (1993) M(r) 92,000 type IV collagenase is increased in plasma of patients with colon cancer and breast cancer. Cancer Res. 53, 140-146.

32. Deutsch, D., and Mertz, B. T. (1970) Plasminogen: purification from human plasma by affinity chromatography. Science 170, 1095-1096.

33. Gonzalez-Gronow, M., and Robbins, K. C. (1984) In vitro biosynthesis of plasminogen in a cell-free system directed by mRNA fractions isolated from monkey liver. Biochemistry 23, 190-196.

34. Shibuya, N., Goldstein, I. J., Broekaert, W. F., Nsimba-Lubaki, M., Peters, B., and Peumans, W. J. (1987) Fractionation of sialylated oligosaccharides, glycopeptides, and glycoproteins on immobilized elderberry (Sambucu nigra L.) bark lectin. Arch. Biochem. Biophys. 254, 1-8.

35. Markwell, M. A. K. (1982) A new solid-state reagent to iodinate proteins. I. Conditions for the efficient labeling of antiserum. Anal. Biochem. 125, 427-432.

36. Nagatsu, T., Hino, M., Fuyamada, H., Hayakawa, T., Nagatsu, T., and Sakakibara, S. (1976) New chromogenic substrates for X-prolyl dipeptidyl aminopeptidase. Anal. Biochem. 74, 466-476.

37. Shibuya-Saruta, H., Kasahara, Y., and Hashimoto, Y. (1996) Human serum dipeptidyl peptidase IV (DPP IV) and its unique properties. J. Clin. Lab. Anal. 10, 435-440.

38. Mann, M., HØjrup, P., and Bleasby, A. J., (1993) Use of mass spectrometric molecular weight information to identify proteins in sequence databases. Biol. Mass Spectrum 22, 338-345.

39. Pappin, D. J. C., HØjrup, P., and Bleasby, A. J. (1993) Rapid identification of proteins by peptide-mass fingerprinting. Curr. Biol. 3, 327-332.

40. Heusen, C., and Dowdle, E. B. (1990) Electrophoretic analysis of plasminogen activators in polyacrylamide gels containing sodium dodecylsulfate and copolimerized substrates. Anal. Biochem. 102, 186-202.

41. Kleiner, D. E., and Stetler-Stevenson, W. G. (1994) Quantitative zymography: detection of picogram quantities of gelatinases. Anal. Biochem. 218, 325-329.

42. Masure, S., Proost, P., Van Damme, J., and Opdenaker, G. (1991) Purification and identification of 91 kDa neutrophil gelatinase. Release by the activating peptide interleukin-8. Eur. J. Biochem. 198, 391-398.

43. Laemli, U. K. (1970) Cleavage of structural proteins during the assembly of the head of bacteriophage T4. Nature 227, 680-685.

44. Towbin, H., Staehlin, T., and Gordon, J. (1979) Electrophoretic transfer of proteins from polyacrylamide gels to nitrocellulose sheets: procedure and some applications. Proc. Natl. Acad. Sci. USA 76, 4350-4355.

45. Wilhelm, S. M., Collier, I. E., Manner, B. L., Eisen, A. Z., Grant, G. A., and Goldberg, G. I. (1989) SV40-transformed human lung fibroblasts secrete a 92 kDa type IV collagenase which is identical to that secreted by normal human macrophages. J. Biol. Chem. 264, 17213-17221.

46. Tso, J. Y., Sun, X., Kao, T., Reece, K. S., and Wu. R. (1985) Isolation and characterization of rat and human glyceraldehyde-3-phosphate dehydrogenase cDNAs: genomic complexity and molecular evolution of the gene. Nucleic Acid Res. 13, 2485-2502.

47. Albini, A., Iwamoto, Y., Kleinman, H. K., Martin, G. R., Aaronson, S. A., Kozlowski, J. M., and McEwan, R. N. (1987) A rapid in vitro assay for quantitating the invasive potential of tumor cells. Cancer Res. 47, 3239-3245.

48. Miles, L. A., and Plow, E. F. (1988) Plasminogen receptors: ubiquitous sites for cellular regulation of fibrinolysis. Fibrinolysis 2, 61-71.

49. Felez, J. (1998) Plasminogen binding to cell surfaces. Fibrinol. Proteol. 12, 183-189.

50. Zeng, F. Y. and Gabius, H. J. (1992) Sialic acid-binding proteins: characterization, biological function and application. Z. Naturforsch. 47, 641-653.

51. Scanlan, M. J., Mohan, Raj, B. K., Calvo, B., Garin-Chesa, P., Sanz-Moncasi, M. P., Healey, J. H., Old, L. J., and Rettig, W. J. (1994) Molecular cloning of fibroblast activation protein alpha, a member of the serine protease family selectively expressed in stromal fibroblasts of epithelial cancers. Proc. Natl. Acad. Sci. USA 91, 5657-5661.

52. Rettig, W. J., Garin-Chesa, P., Healey, J. H., Su, S. L., Ozer, H. L., Schwab, M., Albino, A. P., and Old, L. J. (1993) Regulation and heteromeric structure of the fibroblast activation protein in normal and transformed cells of mesenchymal and neuroectodermal origin. Cancer Res. 53, 3327-3335.

53. Welt, S., Divgi, C. R., Scott, A. M., Garin-Chesa, P., Finn, Graham, M., Carswell, E. A., Cohen, A., Larson, S. M., Old. L. J., and Rettig, W. J. (1994) Antibody targeting in metastatic colon cancer; a phase I study of monoclonal antibody F19 against a cell-surface protein of reactive tumor stromal fibroblasts. J. Clin. Oncol. 12, 1193-1203.

54. Hoosein, N. M., Boyd, D. D., Hollas, W. J., Mazar, A., Henkin, J., and Chung, L. W. K. (1991) Involvement of urokinase and its receptor in the invasiveness of human prostatic carcinoma cell lines. Cancer Comm. 3, 255-264.

55. Wilder, R. L., Case, J. P., Crofford, L. J., Kumkumian, G. K., Lafyatis, R., Remmers, E. F., Sano, H., Sternberg, E. M., and Yocum, D. E. (1991) Endothelial cell and the pathogenesis of rheumatoid arthritis in humans and streptococcal cell wall arthritis in Lewis rats. J. Cell Biochem. 45, 162-166.

56. Saksela, O. and Rifkin, D. B. (1988) Cell-associated plasminogen activation: regulation and physiological functions. Ann. Rev. Cell Biol. 4, 93-126.

57. Ware, J. L., Paulson, D. F., Mickey, G. H., and Webb, K. S. (1982) Spontaneous metastasis of cells of the human prostate carcinoma cell line PC-3 in athymic nude mice. J. Urol. 128, 1064-1067.

58. Hatton, M. W., Southward, S., and Ross-Quellet, B. (1994) Catabolism of plasminogen glycoforms I and II in rabbits: relationship to plasminogen synthesis by the rabbit liver in vitro. Metabolism 43, 1430-1437.

59. Hayes, M. and Castellino, F. J. (1979) Carbohydrate of the human plasminogen variants. I. Structure of the asparagine-linked oligosaccharide unit. J. Biol. Chem. 254, 8722-8726.

60. Hayes, M. and Castellino, F. J. (1979) Carbohydrate of the human plasminogen variants. II. Structure of the O-glycosidically linked oligosaccharide unit. J. Biol. Chem. 254, 8727-828.

61. Takada, Y., Makino, Y., and Takada, A. (1985) Glu-plasminogen I and II: their activation by urokinase and streptokinase in the presence of fibrin and fibrinogen. Thromb. Res. 39, 289-296.

62. Hatton, M. W., Day, S., Ross, B., Southward, S. M. R., DeReske, M., and Richardson, M. (1999) Plasminogen II accumulates five times faster than plasminogen I at the site of a balloon de-endothelializing injury in vivo to the rabbit aorta: comparison with other hemostatic proteins. J. Lab. Clin. Med. 134, 260-266.

63. Barlati, S., De Petro G., Bona, C., Paracini, F., and Tonelli, M. (1995) Phosphorylation of human plasminogen activators and plasminogen. FEBS Letters 363, 170-174.

64. Takahashi, K., Kwaan, C. H., Ikeo, K., and Koh, E. (1992) Phosphorylation of a surface receptor bound urokinase-type plasminogen activator in a human metastatic carcinomatous cell line. Biophys. Res. Commun. 182, 1466-1472.

65. Mastronicola, M. R., Stopelli, M. P., Migliaccio, A., Auriocchio, F., and Blasi, F. (1990) Serine phosphorylation of biosynthetic pro-urokinase from human tumor cells. FEBS Letters 266, 109-114.

66. Franco, P., Massa, O., Garcia-Rocha, M., Chiaradonna, F., Iaccarino, C., Correas, I., Mendes, E., Avila, J., Blasi, F., and Stopelli, M. P. (1998) Protein kinase C-dependent in vivo phosphorylation of prourokinase leads to the formation of a receptor competitive antagonist. J. Biol. Chem. 273, 27734-27740.

67. Nagy, J. A., Brown, L. F., Senger, D. R., Lanir, N., Van De Water, L., Dvorak, A. M., and Dvorak, H. F. (1989) Pathogenesis of tumor stroma generation: a critical role for leaky blood vessels and fibrin deposition. J. Natl. Cancer Inst. 62, 1459-1472.

68. Lijnen, R. H., VanHoef, B., Lupu, F., Moons, L., Carmeliet, P., and Collen, D. (1998) Function of the plasminogen/plasmin and matrix metalloproteinase systems after vascular injury in mice with targeted inactivation of fibrinolytic system genes. Thromb. Vasc. Biol. 18, 1035-1045.

69. Fleisher, B. (1994) CD26: a surface protease involved in T-cell activation. Immunol. Today 15, 180-184.

70. Cheng, H.-C., Abdel-Ghany, M., Elble, R. C., and Pauli, B. U. (1998) Lung endothelial dipeptidyl peptidase IV promotes adhesion and metastasis of rat breast cancer cells via tumor cell surface-associated fibronectin. J. Biol. Chem. 273, 24207-24215.

71. Felder, C. C., Ma, A., Liotta, L. A., and Kohn, E. C. (1991) The antiproliferative and antimetastatic compound L-651,482 inhibits muscarinic acetylcholine receptor-stimulated calcium influx and arachidonic acid release. J. Pharmacol. Exp. Ther. 257, 967-971.

72. Hupe, D. J., Boltz, R., Cohen, C. J., Felix, J., Ham, E., Miller, D., Soderman, D., and Van Skiver, D. (1991) The inhibition of receptor-mediated and voltage-dependent calcium entry by the antiproliferative L-651,482. J. Biol. Chem. 266, 10136-10142.

TABLE I
Binding of Pg 2 glycoforms to 1-LN cells¹

| | | | Bound |
|---|---|---|---|
| Glycoform | Distribution (%) | K_{d} (nM) | (Molecules × 10⁵/cell) |
| Pg 2α | 16.60 ± 2.35 | 24.3 ± 1.33 | 13.0 ± 4.6 |
| Pg 2β | 13.80 ± 1.63 | 7.4 ± 1.86 | 8.3 ± 1.3 |
| Pg 2γ | 22.10 ± 2.17 | 10.6 ± 1.43 | 8.6 ± 1.7 |
| Pg 2δ | 30.92 ± 3.41 | 13.6 ± 1.04 | 18.1 ± 2.4 |
| Pg 2ε | 13.08 ± 1.16 | 3.8 ± 0.83 | 9.4 ± 0.8 |

| | | |
|---|---|---|
| P2 2φ | 3.50 ± 0.85 | No Binding |

¹1-LN cell monolayers in 96 well strip culture plates (2.0 × 10⁴ cells/well) were incubated with serum-free RPMI 1640 in the presence of increasing concentrations of ¹²⁵I-labeled Pg 2 glycoforms.
The assays and calculations of distribution of Pg 2 glycoforms and binding parameters were performed as described under Experimental Procedures. Data shown represent the means ± SD from experiments performed in triplicate.

TABLE II
MMP-9 Activity in Serum-Free Culture Medium of 1-LN Cells
Incubated with Pg 2 Glycoforms¹

| | | Active MMP-9 (ng/ml) | | | | | |
|---|---|---|---|---|---|---|---|
| SFM+ | None | Pg 2α | Pg 2β | Pg 2γ | Pg 2δ | Pg 2ε | Pg 2φ |
| None | 0.58 ± 0.12 | 0.59 ± 0.16 | 0.59 ± 0.13 | 0.60 ± 0.17 | 0.62 ± 0.18 | 1.80 ± 0.21 | 0.63 ± 0.14 |
| 6-AHA | 0.61 ± 0.14 | 0.59 ± 0.17 | 0.5 ± 0.13 | 0.63 ± 0.21 | 0.57 ± 0.14 | 1.82 ± 0.23 | 0.57 ± 0.11 |
| L-lac | 0.15 ± 0.03 | 0.11 ± 0.02 | 0.17 ± 0.04 | 0.15 ± 0.03 | 0.18 ± 0.04 | 0.14 ± 0.05 | 0.11 ± 0.02 |
| DPP IV-Ab | nd² | nd | nd | 0.58 ± 0.12 | 0.56 ± 0.11 | 0.49 ± 0.14 | nd |

¹1-LN cell monolayers in 48 well culture plates (1.7 × 10⁶ cells/well) were incubated with serum-free RPMI 1640 in the absence or presence of purified Pg 2 glycoforms (0.1 μM) in a volume of 0.3 ml at 37° C. for 24 h. The effect of native Pg 2 in the presence of 6-AHA (100 nM), L-lactose (100 mM) or aprotinin (1 μM) was also assessed.
Anti-DPP IV and anti-u-PA IgGs were used at final concentrations of 50 and 100 μg/ml, respectively. The medium was collected and aliquots (50 μl) were assayed for active MMP-9). Values represent the mean ± S.D. of three separate experiments. The statistical significance of differences between means was evaluated by Student's t test.
²nd: not determined.

TABLE III
Effect of Pg 2 glycoforms in the invasive study of 1-LN cells in vitro¹

| | Relative Invasion | | | |
|---|---|---|---|---|
| | (Number of Cells/Field) | | | |
| | Serum-Free | | | |
| Ligand | Medium | +Anti-DPP IV IgG | +Anti-u-PA IgG | +Anti-MMP-9 IgG |
| None | 8.3 ± 2.6 | 9.4 ± 3.2 | 5.8 ± 1.5 | 2.6 ± 1.3 |
| Pg 2α | 13.5 ± 3.4 | nd² | nd | nd |
| Pg 2β | 9.4 ± 2.1 | nd | nd | nd |
| Pg 2γ | 7.8 ± 1.6 | 4.6 ± 2.7 | 5.3 ± 2.8 | 2.1 ± 1.2 |
| Pg 2δ | 9.2 ± 2.5 | 10.4 ± 3.1 | 7.8 ± 2.6 | 1.8 ± 1.0 |
| Pg 2ε | 46.6 ± 5.4 | 3.1 ± 1.6 | 2.1 ± 1.3 | 1.5 ± 1.0 |
| Pg 2φ | 6.8 ± 1.3 | nd | nd | nd |

¹1-LN cells (at a cell density of 1 × 10⁵) were added to a modified Boyden chamber containing an 8 μm pore filter coated with Matrigel (12 μg/filter) in the absence or presence of purified Pg 2 glycoforms (0.1 μM) as described under Experimental Procedures.
Anti-DPP IV, anti-uPA or anti-MMP-9 IgGs were used at final concentrations of 50, 100, and 20 μg/ml, respectively. After incubation at 37° C. for 24 h, filters were excised, non-invading cells were removed from the top surface of the menbrane, stained with Cyto-Quik ™, and invading cells were enumerated by using an ocular micrometer and counting a minimum of five high-powered fields.
Data shown represent the means ± SD from experiments performed in triplicate.
²nd: note determined.

TABLE IV
Function of Pg 2 glycoforms on the surface of 1-LN cells

| | BINDING | | [Ca²⁺]ᵢ | MMP-9 EXPRESSION | |
|---|---|---|---|---|---|
| | +6-AHA | +L-lactose | Increase | Secretion¹ | mRNA² |
| Pg 2α | No | Yes | No | No | No |
| Pg 2β | No | Yes | No | No | No |
| Pg 2γ | Yes | No | Yes | No | No |
| Pg 2δ | Yes | No | Yes | No | No |
| Pg 2ε | Yes | No | Yes | Yes | Yes |
| Pg 2φ | No binding | | | | |

¹Data are shown in Table II and FIGS. 6 and 7.
²Data are shown in FIG. 8.

All documents cited above are hereby incorporated in their entirety by reference. From the foregoing, it will be obvious to those skilled in the art that various modifications in the abovedescribed methods, and compositions can be made without departing from the spirit and scope of the invention. Accordingly, the invention may be embodied in other specific forms without departing from the spirit or essential characteristics thereof. Present embodiments and examples, therefore, are to be considered in all respects as illustrative and not restrictive, and all changes which come within the meaning and range of equivalency of the claims are therefore intended to be embraced therein. All documents referred to herein are hereby incorporated by reference.

We claim:

1. A composition for use in inhibiting metastasis comprising:
a) a CD26 antagonist, plasminogen antagonist, ADA antagonist and/or angiostatin allosteric promoter, and
b) a suitable carrier.

2. The composition of claim 1, wherein the CD26 antagonist, plasminogen antagonist, ADA antagonist and/or angiostatin allosteric promoter are selected from the group consisting of antibodies, antibody fragments, enzymes, peptides and oligonucleotides.

3. The composition of claim 1, wherein the CD26 antagonist, plasminogen antagonist, ADA antagonist and/or angiostatin allosteric promoter is a conjugate of an anti-tumor agent that does not bind to CD26 or plasminogen and a compound that does bind to CD26 or plasminogen.

4. The composition of claim 1, wherein the CD26 antagonist, plasminogen antagonist, ADA antagonist and/or angiostatin allosteric promoter is an antibody or an antibody fragment.

5. The composition of claim 4, wherein the antibody is a monoclonal antibody or antibody fragment thereof.

6. The composition of claim 4, wherein the antibody is a humanized antibody or antibody fragment thereof.

7. The composition of claim 1, wherein the CD26 antagonist, plasminogen antagonist, ADA antagonist and/or angiostatin allosteric promoter are present in or conjugated onto a liposome or microparticle that is of a suitable size for intraveneous administration but that lodges in capillary beds.

8. The composition of claim 1, further comprising an anti-tumor agent that does not bind to CD26 or plasminogen.

9. The composition of claim 1, further comprising an anti-angiogenesis agent.

10. A method of inhibiting tumor metastasis, comprising administering to a patient in need of treatment thereof an effective, metastasis inhibiting amount of a CD26 antagonist, plasminogen antagonist, ADA antagonist and/or angiostatin allosteric promoter.

11. The method of claim 10, wherein the CD26 antagonist, plasminogen antagonist, ADA antagonist and/or angiostatin allosteric promoter is a compound selected from the group consisting of antibodies, antibody fragments, enzymes, peptides and oligonucleotides.

12. The method of claim 10, wherein the CD26 antagonist, plasminogen antagonist, ADA antagonist and/or angiostatin allosteric promoter is a conjugate of an anti-tumor agent that does not bind to CD26 and a CD26 antagonist and/or angiostatin allosteric promoter.

13. The method of claim 10, wherein the CD26 antagonist, plasminogen antagonist, ADA antagonist and/or angiostatin allosteric promoter is an antibody or an antibody fragment.

14. The method of claim 13, wherein the antibody is a monoclonal antibody or antibody fragment thereof.

15. The method of claim 13, wherein the antibody is a humanized antibody or antibody fragment thereof.

16. The method of claim 10, wherein the CD26 antagonist, plasminogen antagonist, ADA antagonist and/or angiostatin allosteric promoter are present in or conjugated onto a liposome or microparticle that is of a suitable size for intraveneous administration but that lodges in capillary beds.

17. The method of claim 10, further comprising administering an anti-tumor agent that does not bind to CD26 or plasminogen.

18. The method of claim 11, wherein the CD26 antagonist, plasminogen antagonist, ADA antagonist and/or angiostatin allosteric promoter is administered intravenously, intramuscularly, intradermally or subcutaneously.

19. A method of screening a test compound for its ability to inhibit metastasis comprising:
i) contacting the test compound with CD26 under conditions such that angiostatin would bind to the CD26 in the absence of the test compound, and
ii) determining the binding affinity of the compound to CD26.

20. The method of claim 19 wherein the compound bears a detectable label.

21. The method of claim 19 wherein the CD26 is attached to a solid support.

22. The method of claim 19 wherein the CD26 is associated with a lipid membrane.

23. The method of claim 22 wherein the membrane is a membrane of an intact cell.

24. The method of claim 23 wherein the cell naturally expresses CD26.

25. The method of claim 23 wherein the cell has been transformed with one or more nucleic acid sequence that encode CD26.

26. A compound identified in the method of claim 19 as inhibiting metastasis.

27. A compound identified in the method of claim 19 as enhancing the binding of angiostatin to CD26.

28. A method of screening a test compound for its ability to inhibit metastasis comprising:
i) contacting the test compound with a cell that expresses CD26 under conditions such that angiostatin would bind to the CD26 in the absence of the test compound and under conditions such that the Ca⁺² signaling cascade that results in formation of MMP-9 would otherwise occur,
ii) determining the amount of MMP-9 formed after the compound is contacted with the CD26, and
iii) comparing the amount of MMP-9 formed with a baseline amount of MMP-9 formed when no test compound is added.

29. A CD26 antagonist identified in accordance with the method of claim 28.

30. A monoclonal antibody or antibody fragment thereof specific for CD26 that functions as an CD26 antagonist.

31. A monoclonal antibody or antibody fragment thereof that functions as an angiostatin allosteric promoter.

32. A monoclonal antibody or antibody fragment thereof that functions as a plasminogen antagonist.

33. A monoclonal antibody or antibody fragment thereof that functions as an ADA antagonist.
